(19) United States
(12) Patent Application Publication
(10) Pub. No.: US 2021/0338678 A1
(43) Pub. Date: Nov. 4, 2021

(54) BIOACTIVE VITAMIN COMBINATIONS

(71) Applicant: BioVit, Inc., San Diego, CA (US)

(72) Inventor: Sheldon Blake Zablow, San Diego, CA (US)

(73) Assignee: BioVit, Inc., San Diego, CA (US)

(21) Appl. No.: 17/246,494

(22) Filed: Apr. 30, 2021

Related U.S. Application Data
(60) Provisional application No. 63/018,443, filed on Apr. 30, 2020.

(51) Int. Cl. A61K 31/519; A61K 31/714; A61K 31/573; A61K 45/06; A61K 38/28; A61K 38/26; A61K 31/522; A61K 31/4985; A61K 31/7042; A61K 31/7048; A61K 31/513; A61K 31/155; A61K 31/4439; A61K 31/137; A61K 31/7036; A61K 31/64; A61K 33/00; A61K 31/7032; A61K 31/65; A61K 31/341; A61K 31/426; A61K 31/5575; A61K 31/138; A61K 31/5513; A61K 31/37; A61K 31/167; A61K 31/58; A61K 31/665; A61K 38/14; A61K 31/5383; A61K 31/4164; A61K 31/36; A61K 31/19; A61K 39/235; A61K 39/29; A61K 39/00; A61K 39/145; A61K 39/285; A61K 39/04; A61K 39/20; A61K 39/215

(52) U.S. Cl. CPC A61K 31/519; A61K 39/29; A61K 31/573; A61K 45/06; A61K 38/28; A61K 38/26; A61K 31/522; A61K 31/4985; A61K 31/7042; A61K 31/7048; A61K 31/513; A61K 31/155; A61K 31/4439; A61K 31/137; A61K 31/7036; A61K 31/64; A61K 33/00; A61K 31/7032; A61K 31/65; A61K 31/341; A61K 31/426; A61K 31/5575; A61K 31/138; A61K 31/5513; A61K 31/37; A61K 31/167; A61K 31/58; A61K 31/665; A61K 38/14; A61K 31/5383; A61K 31/4164; A61K 31/36; A61K 31/19; A61K 39/235; A61K 39/292; A61K 39/0016; A61K 39/0015; A61K 39/145; A61K 39/285; A61K 39/04; A61K 39/20; A61K 39/215; A61K 31/714

(57) ABSTRACT

The present invention describes bioactive vitamin combinations that can be used in combination with other bioactive compounds, such as active drug ingredients or active vaccine components, to increase their therapeutic effects. The bioactive vitamin combinations comprise therapeutically effective amounts of L-Methylfolate, Adenosylcobalamin and Methylcobalamin.

### CROSS-REFERENCE TO RELATED APPLICATIONS

This application claims priority from U.S. Provisional Application Ser. No. 63/018,443 filed on Apr. 30, 2020, which is incorporated herein by reference in its entirety.

### STATEMENT REGARDING FEDERALLY SPONSORED RESEARCH OR DEVELOPMENT

Not Applicable.

### FIELD

The present invention relates to bioactive vitamin supplements that are used in combination with other bioactive compounds to increase their therapeutic effects.

### INTRODUCTION

Over the past fifty years, increasingly processed food has had its natural bioactive vitamins stripped out while having the synthetic forms substituted back in through fortification and supplementation. These artificial vitamins are poorly made, poorly absorbed, and poorly utilized, eventually being just flushed away unused. Energy is required by every cell to complete all the other thousands of biochemical reactions in the most efficient manner possible, as well as to adequately react to the outside stimuli. It is known that low intake or utilization of vitamin B12 and folate (vitamin B9) causes reduced cellular energy, which slows cognitive function, increases fatigue, worsens sleep, and causes mood disturbances. Moreover, a deficiency of either increases chronic inflammation through DNA epigenetic methylation, which contributes to the onset of most major illnesses. B12 and folate deficiency also hamper the elimination of homocysteine. A buildup of homocysteine causes increased blood viscosity, blood clots, inflammation, arterial damage, unhealthy aging and other medical problems. Finally, therapeutic efficiency of many drugs depend on the micronutrient status of the individual, in particular, available cellular levels of vitamin B12 and folate (vitamin B9) as these vitamins are critical co-factors in multiple cellular reactions. All these problems can arise in older persons due to age-related reduction in absorption and bioavailability of vitamins B12 and B9, or due to medication-induced deficiency (multiple prescribed medications can reduce absorption of vitamins B12 and B9 from food).

### SUMMARY

Given the important role of vitamins B12 and B9 in maintaining cellular energy status and control over multiple cellular processes, it was determined that deficiency in bioactive forms of vitamins B12 and B9 can lead decrease efficiency of other bioactive compounds, such as drugs or vaccines. Conversely, supplementation with optimal, bioactive forms of vitamins B12 and B9 can boost cellular responses to other bioactive compounds, such as drugs or vaccines, and increase efficiency of these compounds.

The present teachings include compositions comprising a therapeutically effective amount of: 1) L-Methylfolate, 2) Adenosylcobalamin, 3) Methylcobalamin, and 4) at least one bioactive compound or a pharmaceutically acceptable salt thereof, wherein the bioactive compound does not comprise a vitamin or an elemental metal.

In accordance with a further aspect, the bioactive compound of the composition comprises an active ingredient of a drug or an active component of a vaccine.

In accordance with yet another aspect, the drug is selected from the group consisting of a diabetes therapeutic, gastric acidity therapeutic, hormone replacement therapeutic, oral contraceptive, corticosteroid, antibiotic, antiseizure therapeutic, tumor vaccines, HIV therapeutic, antidepressant, cholesterol therapeutic, gout therapeutic, blood pressure therapeutic, osteoporosis therapeutic, antipsychotic therapeutic, constipation therapeutic, anti-Parkinson's therapeutic, pain therapeutic, oral therapeutic, topical therapeutic, wound therapeutic, nutritional aid, cancer therapeutic, infertility therapeutic, dementia therapeutic, macular degeneration therapeutic, fibromyalgia therapeutic, and any combination thereof.

In accordance with yet another aspect, the claimed composition comprises L-Methylfolate from 500 μg to 1500 μg, Adenosylcobalamin from 1000 μg to 2500 μg and Methylcobalamin from 1000 μg to 2500 μg.

In accordance with yet another aspect, the diabetes therapeutic is selected from the group consisting of insulin, oral antihyperglycemic drugs, sulfonylureas, short-acting insulin secretagogues, biguanides, thiazolidinediones, alpha-glucosidase inhibitors, dipeptidyl peptidase-4 inhibitors, sodium-glucose co-transporter 2 inhibitors, dopamine agonist, injectable antihyperglycemic drugs, glucagon-like peptide-1 (GLP-1) receptor agonists, amylin analog, adjunctive drug therapy, Chlorpropamide, Dapagliflozin, Canagliflozin, Ertugliflozin, Bromocriptin, Empagliflozin, Rosiglitazone, Lixisenatide, Pioglitazone, Repaglinide, liraglutide, Glimepiride, Linagliptin, albiglutide, Tolbutamide, pramlintide, epinephrine, Saxagliptin, Semaglutide, Sitagliptin, dulaglutide, Nateglinide, Alogliptin, Metformin, Glipizide, exenatide, Glyburide, Acarbose, Miglitol, and any combination thereof.

In accordance with yet another aspect, the gastric acidity therapeutic is selected from the group consisting of proton pump inhibitors, H2 blockers, histamine H2 antagonists, antacids, prostaglandins, sucralfate, sodium bicarbonate, esomeprazole, pantoprazole, lansoprazole, theophylline, tetracycline, rabeprazole, misoprostol, omeprazole, nizatidine, Ranitidine, famotidine, cimetidine, Sucralfate, lidocaine, phenytoin, warfarin, diazepam, digoxin, and any combination thereof.

In accordance with yet another aspect, the hormone replacement therapeutic is selected from the group consisting of conjugated equine or synthetic estrogens, estradiol, medroxyprogesterone acetate, progesterone, natural progesterone, synthetic progesterone, norethindrone, drospirenone, levonorgestrel, megastrol, selective estrogen receptor modulators, tamoxifen, raloxifene, bazedoxifene, and any combination thereof.

In accordance with yet another aspect, the oral contraceptive therapeutics is selected from the group consisting of gonadotropin-releasing hormone inhibitors, pituitary hormone inhibitors, estradiol, ethinyl estradiol, estrogen with progestin, estrogen without progestin, progestins, Norgestimate, etonogestrel, desogestrel, levonorgestrel, norethindrone, norethindrone acetate, ethynodiol diacetate, dienogest, drospirenone, progesterone, and any combination thereof.

In accordance with yet another aspect, the corticosteroid therapeutic is selected from the group consisting of Class I corticosteroids, Class II corticosteroids, Class III corticosteroids, Class IV corticosteroids, Class V corticosteroids, Class VI corticosteroids, Class VII corticosteroids, Betamethasone dipropionate, Betamethasone valerate, Clobetasol propionate, Diflorasone diacetate, Halobetasol propionate, Amcinonide, Desoximetasone, Fluocinonide, Fluocinolone acetonide, Halcinonide, Mometasone furoate, Fluticasone propionate, Triamcinolone acetonide, Flurandrenolide, Desonide, Hydrocortisone, Hydrocortisone acetate, Hydrocortison acetate and pramoxine hydrochloride, Hydrocortisone butyrate, Hydrocortisone valerate, Alclometasone dipropionate, Flumethasone pivalate, hydrocortisone types, acetonides and related substances, betamethasone types, esters, halogenated esters, labile prodrug esters, and any combination thereof.

In accordance with yet another aspect, the antibiotics is selected from the group consisting of sulfa drugs, cephalosporins, macrolides, penicillin derivatives, quinolones, tetracycline derivatives, aminoglycosides, trimethoprim, quinupristin/dalfopristin, piperacillin/tazobactam, Penicillin G procaine, clarithromycin, nitrofurantoin, ciprofloxacin, telithromycin, Metronidazole, levofloxacin, erythromycin, Theophylline, Gemifloxacin, Tetracycline, azithromycin, Delafloxacin, Eravacycline, moxifloxacin, Dalbavancin, Amoxicillin, Fidaxomicin, Tigecycline, ceftriaxone, Minocycline, Rifapentine, Clindamycin, Ceftazidime, Oritavancin, Norfloxacin, Doxycycline, Cefuroxime, Tobramycin, Ceftibuten, Gentamicin, Cefotaxime, Vancomycin, Telavancin, Daptomycin, Cephalexin, Fosfomycin, tedizolid, Aztreonam, Nafcillin, Phenytoin, Ertapenem, Cefazolin, Isoniazid, Doripenem, rifabutin, Meropenem, Linezolid, ofloxacin, Cefoxitin, Oxacillin, Warfarin, Neomycin, Rifampin, Cefepime, Digoxin, and any combination thereof.

In accordance with yet another aspect, the antiseizure therapeutic is selected from the group consisting of broad-spectrum antiseizure drugs, focal-onset seizure drugs, generalized-onset tonic-clonic seizure drugs, epileptic spasms drugs, atonic seizures drugs, myoclonic seizures drugs, juvenile myoclonic epilepsy drugs, febrile seizure drugs, drugs for seizures due to alcohol withdrawal, anticonvulsants, Eslicarbazepine, Levetiracetam, oxcarbazepine, Acetazolamide, Carbamazepine, phenobarbital, Fosphenytoin, Ethosuximide, lamotrigine, Zonisamide, lacosamide, topiramate, vigabatrin, clonazepam, pregabalin, gabapentin, felbamate, phenytoin, Ezogabine, tiagabine, clobazam, mephobarbital, primidone, valproic acid, and any combination thereof.

In accordance with yet another aspect, the vaccine is selected from the group consisting of Adenovirus; Anthrax, including but not limited to AVA; Cholera, including but not limited to Vaxchora; Diphtheria, including but not limited to DTaP, Td, DT, Tdap, DTaP-IPV, DTaP-HepB-IPV, DTaP-IPV/Hib; Hepatitis A, including but not limited to HepA, HepA-HepB; Hepatitis B including but not limited to HepB, DTaP-HepB-IPV, HepA-HepB; Haemophilus influenzae type b (Hib) including but not limited to Hib, DTaP-IPV/Hib; Human Papillomavirus (HPV) including but not limited to HPV9/9vHPV; Seasonal Influenza (Flu) including but not limited to IIV, LAIV; Japanese Encephalitis including but not limited to JE; Measles including but not limited to MMR, MMRV; Meningococcal including but not limited to MenACWY, MenB; Mumps including but not limited to MMR, MMRV; Pertussis including but not limited to DTaP, Tdap, DTaP-IPV, DTaP-HepB-IPV, DTaP-IPV/Hib; Pneumococcal including but not limited to PCV13, PPSV23; Polio including but not limited to Polio, DTaP-IPV, DTaP-HepB-IPV, DTaP-IPV/Hib; Rabies including but not limited to Rabies; Rotavirus including but not limited to RV1, RV5; Rubella including but not limited to MMR, MMRV; Shingles including but not limited to ZVL, RZV; Smallpox including but not limited to Vaccinia; Tetanus including but not limited to DTaP, Td, DT, Tdap, DTaP-IPV, DTaP-HepB-IPV, DTaP-IPV/Hib; Tuberculosis; Typhoid Fever including but not limited to Typhoid Oral, Typhoid Polysaccharide; Varicella including but not limited to VAR, MMRV; Yellow Fever including but not limited to YF; SARS-CoV-2, and any combination thereof.

In accordance with yet another aspect, the HIV therapeutic is selected from the group consisting of antiretroviral therapy (ART), antiretrovirals, nucleoside reverse transcriptase inhibitors (NRTIs), nucleotide reverse transcriptase inhibitors (nRTIs), non-nucleoside reverse transcriptase inhibitors (NNRTIs), protease inhibitors (PIs), entry inhibitors (EIs), post-attachment inhibitors, integrase inhibitors, cyclophosphamide, clarithromycin, Fosamprenavir, Emtricitabine, ketoconazole, itraconazole, Elvitegravir, Dolutegravir, Rilpivirine, Enfuvirtide, Bictegravir, Raltegravir, didanosine, Saquinavir, Atazanavir, Lamivudine, Nelfinavir, Doravirine, Zidovudine, Etravirine, Tipranavir, Nevirapine, Ibalizumab, Stavudine, Maraviroc, estradiol, ritonavir, Darunavir, Tenofovir, Efavirenz, methadone, cisapride, warfarin, fentanyl, Abacavir, and any combination thereof.

In accordance with yet another aspect, the antidepressant therapeutic is selected from the group consisting of selective serotonin reuptake inhibitors (SSRIs), serotonin modulators (5-HT₂ blockers), serotonin-norepinephrine reuptake inhibitors, norepinephrine-dopamine reuptake inhibitors, heterocyclic antidepressants, monoamine oxidase inhibitors (MAOIs), melatonergic antidepressant, ketamine-like drugs, dextromethorphan, tranylcypromine, pseudoephedrine, levomilnacipran, chlorpromazine, desvenlafaxine, Protriptyline, isocarboxazid, carbamazepine, nortriptyline, clomipramine, vortioxetine, theophylline, escitalopram, Trimipramine, desipramine, propranolol, fluvoxamine, venlafaxine, Mirtazapine, amphetamine, paroxetine, sertraline, selegiline, vilazodone, phenelzine, citalopram, fluoxetine, imipramine, meperidine, metoprolol, duloxetine, esketamine, Trazodone, Bupropion, clozapine, ketamine, warfarin, Doxepin, and any combination thereof.

In accordance with yet another aspect, the cholesterol therapeutic is selected from the group consisting of statins, bile acid sequestrants, cholestyramine, cholesterol absorption inhibitors, PCSK9 monoclonal antibodies, dietary supplements, drugs for homozygous familial hypercholesterolemia, fibrates, omega-3 fatty acids, Apo CIII inhibitor, nicotinic acid (niacin), cholesterol ester transport protein (CETP) inhibitors, Pitavastatin, Rosuvastatin, Atorvastatin, cyclosporine, Pravastatin, gemfibrozil, Bezafibrate, Fluvastatin, Colesevelam, Simvastatin, Lomitapide, tacrolimus, Mipomersen, alirocumab, evolocumab, Lovastatin, Colestipol, Metformin, Ezetimibe, warfarin, digoxin, niacin, and any combination thereof.

In accordance with yet another aspect, the gout therapeutic is selected from the group consisting of nonsteroidal anti-inflammatory drugs (NSAIDs), corticosteroids, uriosuric therapy, uriosuric agents, clarithromycin, mercaptopurine, nitroglycerin, acetazolamide, azathioprine, prednisolone, cyclosporine, allopurinol, pegloticase, prednisone, colchicine, probenecid, febuxostat, losartan, anakinra, and any combination thereof.

In accordance with yet another aspect, the blood pressure therapeutic is selected from the group consisting of adrenergic modifiers, central alpha-2-agonists, postsynaptic alpha-1-blockers, peripheral-acting adrenergic blockers, angiotensin-converting enzyme (ACE) inhibitors, angiotensin II receptor blockers (ARBs), beta-blockers, calcium channel blockers, dihydropyridines, nondihydropyridines, direct renin inhibitors, direct vasoldilators, diuretics, loop diuretics, potassium-sparing diuretics, thiazide-type diuretics, Perindopril erbumine, perindopril, Hydrochlorothiazide, Methyclothiazide, Ethacrynic acid, Spironolactone, chlorthalidone, Chlorothiazide, Trandolapril, triamterene, Candesartan, Telmisartan, Propranolol, Nicardipine, Nisoldipine, Lisinopril, Amlodipine, carvedilol, Furosemide, nifedipine, Benazepril, Irbesartan, Eprosartan, Guanfacine, Felodipine, Olmesartan, acebutolol, metoprolol, Azilsartan, methyldopa, bisoprolol, eplerenone, verapamil, Captopril, amiloride, Quinapril, diltiazem, betaxolol, Enalapril, torsemide, Valsartan, Terazosin, Aliskiren, carteolol, Nebivolol, Doxazosin, minoxidil, Clonidine, Ramipril, Losartan, Prazosin, atenolol, lithium, Nadolol, Timolol, bumetanide, indapamide, hydralazine, fosinopril, moexipril, and any combination thereof.

In accordance with yet another aspect, the osteoporosis therapeutic is selected from the group consisting of calcium and vitamin D supplements, antiresorptive drugs, bisphosphonates, hormone replacement therapy, selective estrogen receptor modulators, activator of nuclear factor kappa-B ligand (RANKL) inhibitor, anabolic agents, parathyroid hormone (PTH) analogues, romosozumab, medroxyprogesterone, cholecalciferol, Zoledronic acid, ergocalciferol, abaloparatide, rosiglitazone, teriparatide, pioglitazone, tetracycline, Ibandronate, Alendronate, Risedronate, Raloxifene, calcitriol, calcitonin, denosumab, heparin, and any combination thereof.

In accordance with yet another aspect, the antipsychotic therapeutic is selected from the group consisting of dopamine-2 blockers, second-generation psychotics (SGAs), Diphenhydramine, Trihexyphenidyl, norepinephrine, Brexpiprazole, Paliperidone, Aripiprazole, Fluphenazine, Thiothixene, ziprasidone, Iloperidone, Benztropine, risperidone, Cariprazine, haloperidol, Lurasidone, olanzapine, Clonazepam, quetiapine, Lorazepam, clozapine, Loxapine, Pimozide, Lithium, and any combination thereof.

In accordance with yet another aspect, the constipation therapeutic is selected from the group consisting of osmotic laxatives, bulking agents, osmotic agents, secretory or stimulant cathartics, enemas, emollient agents, peripherally acting mu-opioid receptor antagonists (PAMORAs), magnesium sulfate, Lubiprostone, Linaclotide, lidocaine, Lactulose, Bisacodyl, senna, miralax, psyllium, calcium polycarbophil, methylcellulose, phenolphthalein, anthraquinones, castor oil, ducosate, mineral oil, lactulose, and any combination thereof.

In accordance with yet another aspect, the anti-Parkinson's therapeutic is selected from the group consisting of Carbidopa/levodopa, Amantadine, MAO type B (MOA-B) inhibitors, anticholinergic drugs, dopamine agonists, catechol O-methyltransferase (COMT) inhibitors, oral dopamine agonists, trimethobenzamide, diphenhydramine, Trihexyphenidyl Bromocriptine, Benztropine, risperidone, pramipexole, apomorphine, amphetamine, rasagiline, entacapone, selegiline, olanzapine, rotigotine, quetiapine, ropinirole, tolcapone, clozapine, carbidopa, bisacodyl, levodopa, and any combination thereof.

In accordance with yet another aspect, the pain therapeutic is selected from the group consisting of nonopioid analgesics, opioid analgesics, antidepressants, antiseizure drugs, central nervous system (CNS)-active drugs, adjuvant analgesic drugs, nonsteroidal anti-inflammatory drugs (NSAIDs), aspirin, codeine, guaifenesin, hydrocodone, Choline magnesium trisalicylate, Dextroamphetamine, Dextromethorphan, prochlorperazine, diphenhydramine, Methylphenidate, norepinephrine, Metoclopramide, Mefenamic acid, Oxcarbazepine, Hydromorphone, Dexamethasone, Acetaminophen, Buprenorphine, Carbamazepine, nortriptyline, Indomethacin, lubiprostone, testosterone, Flurbiprofen, desipramine, misoprostol, venlafaxine, Hydroxyzine, pentazocine, Pamidronate, tamsulosin, Prednisone, Ketoprofen, pregabalin, Tizanidine, Meperidine, gabapentin, ziconotide, Naltrexone, diclofenac, duloxetine, Fenoprofen, Ibuprofen, bupropion, ketorolac, Capsaicin, lidocaine, Celecoxib, Phenytoin, Modafinil, lactulose, Oxycodone, Memantine, Piroxicam, Methadone, Oxaprozin, bisacodyl, clonidine, naloxone, Tramadol, Sulindac, Naproxen, morphine, Fentanyl, Baclofen, and any combination thereof.

In accordance with yet another aspect, the oral therapeutic is selected from the group consisting of toothpaste, dental plaque removers, halitosis suppressing treatments, dental disorder treatments, oral disorder treatments, fluoride, gingivitis treatments, baking soda, salt, lidocaine, saline rinses, topical gum infection agents, floss, whitening treatments, hydrogen peroxide, and any combination thereof.

In accordance with yet another aspect, the topical therapeutic is selected from the group consisting of shampoos, conditioners, hair lotions, moisturizers, psoriasis treatments, immunosuppressants, emollients, salicylic acid, coal tar, anthralin, corticosteroids, vitamin D3 analogs, calcineurin inhibitors tazarotene, oral retinoids, cyclosporine, immunomodulatory agents, skin supplements and powders, hair supplements and powders, nail supplements and powder, lip balms, insecticides, keratolytics, astringents, antipruritics, sunscreens, and any combination thereof.

In accordance with yet another aspect, the wound therapeutic is selected from the group consisting of bandages, wound healings, burn dressings, disinfectants, sterilizers, antiseptic agents, iodine solutions, and any combination thereof.

In accordance with yet another aspect, the nutritional use is selected from the group consisting of vegan meat replacement products, vegan fish replacement products, food fortifications, protein powders, protein bars, nutritional supplements, enteral nutrition, medicinal foods, small and large animal medications, small and large animal feeds, veterinary treatments, infant formulas, breastfeeding alternatives, milk powders, concentrated liquids, prediluted liquids, energy drinks, patches, injections, antifungals, and any combination thereof.

In accordance with yet another aspect, the cancer therapeutic is selected from the group consisting of antineoplastic therapy, hormonal therapy, immunotherapy, monoclonal antibodies, interferons, biologic response modifiers, tumor vaccines, cell therapies, retinoids, isocitrate dehydrogenase-2 (IDH2) inhibitors, imatinib, and any combination thereof.

In accordance with yet another aspect, the infertility therapeutics is selected from the group consisting of sperm disorder treatments, Medroxyprogesterone, Cyclophosphamide, Spironolactone, Nitrofurantoin, Sulfasalazine, bicalutamide, Ketoconazole, Chlorambucil, cyproterone, Colchicine, Cimetidine, Clomiphene, Estrogens, ovulatory dysfunction treatments, exogenous gonadotropins, progesterone, gonadorelin, metformin, letrozole, decreased ovarian reserve (DOR) treatments, donor oocytes, estradiol, tubal dysfunction and pelvic lesions treatments, abnormal cervical mucus treatments, controlled ovarian stimulation (COS) treatments, urofollitropin, synthetic human chorionic gonadotropin (hCG), follicle stimulating hormone (FSH), methylprednisolone, and any combination thereof.

In accordance with yet another aspect, the dementia therapeutic is selected from the group consisting of cholinesterase inhibitors, rivastigmine, benztropine, propranolol, galantamine, memantine, donepezil, and any combination thereof.

In accordance with yet another aspect, the macular degeneration therapeutic is selected from the group consisting of lutein, zeaxanthin, antivascular endothelial growth factor (anti-VEGF) drugs, corticosteroids, triamcinolone, bevacizumab, aflibercept, ranibizumab, and any combination thereof.

In accordance with yet another aspect, the fibromyalgia therapeutic is selected from the group consisting of tricyclic antidepressants, nonopioid analgesics, cyclobenzaprine, amitriptyline, acetaminophen, milnacipran, bupivacaine, Pregabalin, duloxetine, trazodone, lidocaine, doxepin, and any combination thereof.

The present teachings also include methods for treating a subject in need thereof with any one of the above-described compositions, the method comprising administering the compositions to the subject.

The present teachings also include kits comprising any one of the above-described compositions and an instruction of how to use the composition.

The present teachings also describe use of any one of the above-described compositions as a medicament.

The present teachings also describe use of any one of the above-described compositions for treating a subject.

These and other features, aspects and advantages of the present teachings will become better understood with reference to the following description, examples and appended claims.

### DRAWINGS

Not Applicable.

### DETAILED DESCRIPTION

### Abbreviations and Definitions

To facilitate understanding of the invention, a number of terms and abbreviations as used herein are defined below as follows.

As used herein, the term “bioactive vitamin” is defined to include any active, substantially bioavailable compound which does not require metabolism in the human body to be therapeutically effective. For example, it is well known by those of skill in the art that folic acid is not readily bioavailable and requires conversion into an active form to be therapeutically effective. Hence, the present innovation does not include folic acid as a bioactive vitamin, but L-methylfolate is included in the definition of “bioactive vitamin”. In the present disclosure, three bioactive vitamins are used: a bioactive form of folic acid (vitamin B9), L-methylfolate, also known as Levomefolic acid, L-5-MTHF, L-5-methyltetrahydrofolate, (6S)-5-methyltetrahydrofolate, and (6S)-5-MTHF; and two bioactive forms of vitamin B12: Adenosylcobalamin (also known as 5′-deoxyadenosylcobalamin) cobamamide, dibencozide, and Methylcobalamin.

As used herein, the term “bioactive compound” is defined to include any pharmaceutically or biologically active compounds capable of inducing, inhibiting or interfering with a physiological activity of a mammal. In preferred embodiments, bioactive compound is an active ingredient of a drug used to diagnose, cure, treat or prevent disease. In some embodiments, bioactive compound does not comprise a vitamin or an elemental metal. In particular embodiments, bioactive compounds can be a small molecule compound, a nucleic acid, a polypeptide, or a functional fragment or variant thereof. In one instance, the agent is a small molecule compound (e.g., a compound having a molecule weight of less than about 1000 Da). In some embodiments, bioactive compounds can be an active component of a vaccine. In some embodiments, bioactive compounds in the combinations described herein include nucleic acids and/or polypeptides. In some embodiments, bioactive compound is an antibody, peptibody, diabody, minibody, a single-chain variable fragment (ScFv), or a functional fragment thereof. In some embodiments, the bioactive compound can be a diabetes therapeutic, gastric acidity therapeutic, hormone replacement therapeutic, oral contraceptive, corticosteroid, antibiotic, antiseizure therapeutic, tumor vaccines, HIV therapeutic, antidepressant, cholesterol therapeutic, gout therapeutic, blood pressure therapeutic, osteoporosis therapeutic, antipsychotic therapeutic, constipation therapeutic, anti-Parkinson's therapeutic, pain therapeutic, oral therapeutic, topical therapeutic, wound therapeutic, nutritional aid, cancer therapeutic, infertility therapeutic, dementia therapeutic, macular degeneration therapeutic, fibromyalgia therapeutic, and any combination thereof.

As used herein, the term “biological activity” refers to the biological effect of a substance on living matter. Accordingly, the terms “biologically active protein” or “polypeptide having and/or mediating biological activity” as used herein relate to proteins or polypeptides that are capable of inducing a biological effect in living cells/organisms that are exposed to said protein or polypeptide.

As used herein, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “pharmaceutically acceptable carrier” refers to a diluent, adjuvant, excipient, or vehicle with which a compound is administered. Such carriers can be sterile liquids, such as water and oils, including those of petroleum, animal, vegetable, or synthetic origin, such as peanut oil, soybean oil, mineral oil, sesame oil and the like, polyethylene glycols, glycerine, propylene glycol, or other synthetic solvents. Water is a preferred carrier when a compound is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. A compound, if desired, can also combine minor amount of wetting or emulsifying agents, or pH buffering agents such as acetates, citrates, or phosphates. Antibacterial agents such as a benzyl alcohol or methyl parabens; antioxidants such as ascorbic acid or sodium bisulfate; chelating agents such as ethylenediaminetetraacetic acid; and agents for the adjustment of tonicity such as sodium chloride or dextrose may also be a carrier. Methods for producing compounds in combination with carriers are known to those of skill in the art.

As used herein, the term “pharmaceutically acceptable salt” includes those salts of a pharmaceutically acceptable compound formed with free amino groups such as those derived from hydrochloric, phosphoric, acetic, oxalic, and tartaric acids, and those formed with free carboxyl groups such as those derived from sodium, potassium, ammonium, calcium, ferric hydroxides, isopropylamine, triethylamine, 2-ethylamino ethanol, histidine, and procaine. If the compound is basic, salts may be prepared from pharmaceutically acceptable non-toxic acids including inorganic and organic acids. Such acids include acetic, benzene-sulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic, and the like. Particularly preferred are besylate, hydrobromic, hydrochloric, phosphoric, and sulfuric acids. If the compound is acidic, salts may be prepared from pharmaceutically acceptable organic and inorganic bases. Suitable organic bases include, but are not limited to, lysine, N,N′-dibenzylethylenediamine, chloroprocaine, choline, diethanolamine, ethylene diamine, meglumine (N-methyl-glucamine) and procaine. Suitable inorganic bases include, but are not limited to, alkaline and earth-alkaline metals such as aluminum, calcium, lithium, magnesium, potassium, sodium, and zinc. Methods for synthesizing such salts are known to those of skill in the art.

The term “therapeutically effective amount” refers to the amount of a therapy (e.g., a combination provided herein or another active agent, such as an diabetes therapeutic, gastric acidity therapeutic, hormone replacement therapeutic, oral contraceptive, corticosteroid, antibiotic, antiseizure therapeutic, tumor vaccines, HIV therapeutic, antidepressant, cholesterol therapeutic, gout therapeutic, blood pressure therapeutic, osteoporosis therapeutic, antipsychotic therapeutic, constipation therapeutic, anti-Parkinson's therapeutic, pain therapeutic, oral therapeutic, topical therapeutic, wound therapeutic, nutritional aid, cancer therapeutic, infertility therapeutic, dementia therapeutic, macular degeneration therapeutic, fibromyalgia therapeutic, and any combination thereof described herein (alternatively defined as a “bioactive compound”)) which is sufficient to accomplish a stated purpose or otherwise achieve the effect for which it is administered. An effective amount can be sufficient to reduce and/or ameliorate the progression, development, recurrence, severity and/or duration of a given disease, disorder or condition and/or a symptom related thereto. An effective amount can be a “therapeutically effective amount” which refers to an amount sufficient to provide a therapeutic benefit such as, for example, the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. A therapeutically effective amount of a composition described herein can enhance the therapeutic efficacy of another therapeutic agent.

The term “regimen” refers to a protocol for dosing and timing the administration of one or more therapies (e.g., combinations described herein or another active agent such as bioactive compound described herein) for treating a disease, disorder, or condition described herein. A regimen can include periods of active administration and periods of rest as known in the art. Active administration periods include administration of combinations and compositions described herein and the duration of time of efficacy of such combinations and compositions. Rest periods of regimens described herein include a period of time in which no compound is actively administered, and in certain instances, includes time periods where the efficacy of such compounds can be minimal. Combination of active administration and rest in regimens described herein can increase the efficacy and/or duration of administration of the combinations and compositions described herein.

The terms “therapies” and “therapy” refer to any protocol(s), method(s), and/or agent(s) that can be used in the prevention, treatment, management, and/or amelioration of a disease, disorder, or condition or one or more symptoms thereof. In certain embodiments the term refers to active agents such as a bioactive compound described herein. The terms “therapy” and “therapy” can refer to anti-viral therapy, anti-bacterial therapy, anti-fungal therapy, anti-cancer therapy, biological therapy, supportive therapy, and/or other therapies useful in treatment, management, prevention, or amelioration of a disease, disorder, or condition or one or more symptoms thereof known to one skilled in the art, for example, a medical professional such as a physician.

The term “patient” or “subject” refers to a mammal, such as a human, bovine, rat, mouse, dog, horse, monkey, ape, goat, sheep, cow, or deer. Generally, a patient as described herein is human.

The terms “inhibition,” “inhibit,” and “inhibiting” refer to a reduction in the activity, binding, or expression of a polypeptide or reduction or amelioration of a disease, disorder, or condition or a symptom thereof. Inhibiting as used here can include partially or totally blocking stimulation, decreasing, preventing, or delaying activation or binding, or inactivating, desensitizing, or down-regulating protein or enzyme activity or binding.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “enhance” refers to an increase or improvement in the function or activity of a protein or cell after administration or contacting with a combination described herein compared to the protein or cell prior to such administration or contact.

The term “administering” refers to the act of delivering a combination or composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arteriole, intradermal, subcutaneous, intraperitoneal, intraventricular, and intracranial administration. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition).

The term “co-administration” refers to administration of two or more agents (e.g., a combination described herein and another active agent such as a bioactive compound described herein). The timing of co-administration depends in part of the combination and compositions administered and can include administration at the same time, just prior to, or just after the administration of one or more additional therapies, and in one non-limiting example, cancer therapies such as chemotherapy, hormonal therapy, radiotherapy, or immunotherapy. The compound of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation). A first compound or agent can also be used Epigenetic Sensitization of a tumor within a patient before administration of a second compound or agent. The compounds described herein can be used in combination with one another, and with other active agents known to be useful in treating cancer.

The term “anti-cancer agent” is used in accordance with its plain ordinary meaning and refers to a composition having anti-neoplastic properties or the ability to inhibit the growth or proliferation of cells. In various embodiments, an anti-cancer agent is a chemotherapeutic agent. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” refers to a therapy or regimen that includes administration of a chemotherapeutic or anti-cancer agent described herein.

### Bioactive Vitamin Combinations

The present invention is directed to compositions comprising bioactive forms of vitamins B12 and B9 that are formulated to boost cellular responses to other bioactive compounds, such as drugs or vaccines, provided in combination with the vitamins. It is known that vitamins B12 and B9 play an important role in maintaining cellular energy status and controlling multiple cellular processes, such as DNA synthesis, epigenetic regulation of gene expression, development of red blood cells, proper functioning of the nervous system and others. Deficiency in B12/B9 can exacerbate multiple pathologies, including cancer and cardiovascular disease.

The B12 molecule exists in essentially four different configurations. The two naturally occurring, bioactive vitamers in animals are called adenosylcobalamin (A-B12) because of its adenosyl attachment, and methylcobalamin (M-B12) because of its methyl attachment. The third is cyanocobalamin (C-B12) having a cyanide addition, an artificial, man-made form of B12 that must be converted in the body into one of the bioactive vitamers before it can be used. The fourth form, hydroxocobalamin (H-B12), having the hydroxyl attachment, is manufactured by bacteria “in vivo” or by bacteria in a factory, and it also must be converted by the body into one of the bioactive vitamers. However, rates of conversion and overall bioavailability of B-12 supplied in the forms of C-B12 or H-B12 can be significantly hampered by genetic mutations, certain medications and compositions of multi-vitamin supplementations. For example, presence of vitamin C together with vitamin B12 in one pill can result in inactivation of vitamin B12. Vitamin B12 in its natural form is present only in foods of animal origin, which is why its deficit is more common among strict vegetarians, populations with a low intake of animal foods or aged individuals. Folic acid (vitamin B9) works in concert with vitamin B12 in several metabolic pathways within the body. L-methylfolate represents the natural, bioactive form of vitamin B9 found in many foods.

There were reported many conditions where a B12 deficiency or B9 deficiency is an etiological factor or at least an aggravating factor. These conditions can be improved by adequate B12 supplementation of these vitamins. Often, the reversal of these conditions requires not only B12/B9 supplementation, but also an adequate cellular response initiated by a specific drug. Thus, supplementation of B12/B9 can work in synergy with prescribed medications to alleviate certain medical conditions. Importantly, to ensure efficient supplementation, B12/B9 should be taken in their natural, bioactive forms as disclosed herein. Importantly, despite the fact that Adenosylcobalamin and Methylcobalamin both represent bioactive forms of B12, they are not identical regarding supporting important metabolic cascades. Adenosylcobalamin is stored in mitochondria and is a major form in cellular tissues, whereas Methylcobalamin is mostly located in cytosol and is observed mostly in blood and in other body fluids. Based on the described differential location, supplementation of both bioactive forms of B12 is desirable for optimal performance of diverse metabolic cascades supported by B12.

Cancer development is among the conditions influenced by B12/B9 deficiency, and the described B12/B9 supplementation would support anti-cancer activities of certain therapeutics. Epigenetic methylation is one of the essential regulators of genetic expression such that reduced methylation decreases suppression of oncogenic genes. B12/folate deficiency caused by any of the multiple risk factors including increased BMI of an individual, certain medications (prescribed hormones, acid regulators and others), age, special diet, can lead to decreased epigenetic methylation and ultimately to increased cancer susceptibility. Normalizing B12/folate levels would correct epigenetic methylation and act synergistically with other prescribed anti-cancer therapeutics to fix disrupted cellular pathways.

Importantly, B12/folate deficiency also leads to increase in homocysteine, since these vitamins are responsible to metabolize it back into methionine. Homocysteine is another contributor to the development of cancer (Wu et al., “Hyperhomocysteinemia is a risk factor for cancer and a new potential tumor marker” Clinica Chimica Acta. 2002 August; 322(1-2):21-8). It is possible that hyperhomocysteinemia and hypomethylation are working synergistically. Both of these conditions increase inflammatory load for an individual, which can lead to development of multiple disorders, such as cancer, osteoarthritis, diabetes, dementia (these disorders are often associated with normal aging process). Adequate supplementation with compositions comprising bioactive forms of vitamins B12 and B9 described herein, can reduce one of the causes for the descried conditions and can work together with the prescribed targeted therapeutics. The use of the bioactive versions of folate and B12 (methylcobalamin and adenosylcobalamin) could produce protective benefits against several age-related disorders. These bioactive forms eliminate concerns about a genetic inability to convert the synthetic molecules, supplied in common vitamin supplements, into bioactive vitamins (i.e., 30-40% of people have some form of MTHFR polymorphism, which prevents such conversion).

Based on the above-described mechanism, a combination comprising a therapeutically effective amount of: 1) L-Methylfolate, 2) Adenosylcobalamin, 3) Methylcobalamin can act synergistically with another bioactive compound to normalize cellular processes. In preferred embodiments, the bioactive compound comprises an active ingredient of a drug or an active component of a vaccine. In some embodiments, the bioactive compound does not comprise a vitamin or an elemental metal.

The following are examples of therapeutics that can act synergistically with the described combination of bioactive vitamins.

Diabetes therapeutics comprise, but are not limited to, insulin, oral antihyperglycemic drugs, sulfonylureas, short-acting insulin secretagogues, biguanides, thiazolidinediones, alpha-glucosidase inhibitors, dipeptidyl peptidase-4 inhibitors, sodium-glucose co-transporter 2 inhibitors, dopamine agonist, injectable antihyperglycemic drugs, glucagon-like peptide-1 (GLP-1) receptor agonists, amylin analog, adjunctive drug therapy, Chlorpropamide (DIABINESE®), Dapagliflozin (FARXIGA®), Canagliflozin (INVOKANA®), Ertugliflozin (ERTUGLIFLOZIN®), Bromocriptine (PARLODEL®), Empagliflozin (JARDIANCE®), Rosiglitazone (AVANDIA®), Lixisenatide (LIXISENATIDE®), Pioglitazone (ACTOS®), Repaglinide (PRANDIN®), liraglutide (VICTOZA®), Glimepiride (AMARYL®), Linagliptin (TRADJENTA®), albiglutide (TANZEUM®), Tolbutamide, pramlintide (SYMLIN®), epinephrine (ADRENALIN®), Saxagliptin (ONGLYZA®), Semaglutide (SEMAGLUTIDE®), Sitagliptin (JANUVIA®), dulaglutide (TRULICITY®), Nateglinide (STARLIX®), Alogliptin (NESINA®), Metformin (GLUCOPHAGE®), Glipizide (GLUCOTROL®), exenatide (BYETTA®), Glyburide (DIABETA®), (GLYNASE®), Acarbose (PRECOSE®), Miglitol (GLYSET®).

Gastric acidity therapeutics comprise, but are not limited to, proton pump inhibitors, H2 blockers, histamine H2 antagonists, antacids, prostaglandins, sucralfate, sodium bicarbonate, esomeprazole (NEXIUM®), pantoprazole (PROTONIX®), lansoprazole (PREVACID®), theophylline (ELIXOPHYLLIN®), tetracycline (ACHROMYCIN V®), rabeprazole (ACIPHEX®), misoprostol (CYTOTEC®), omeprazole (PRILOSEC®), nizatidine (AXID®), Ranitidine (ZANTAC®), famotidine (PEPCID®), cimetidine (TAGAMET®), Sucralfate (CARAFATE®), lidocaine (XYLOCAINE®), phenytoin (DILANTIN®), warfarin (COUMADIN®), diazepam (VALIUM®), digoxin (LANOXIN®).

Hormone replacement therapeutics comprise, but are not limited to, conjugated equine or synthetic estrogens, estradiol, medroxyprogesterone acetate (PROVERA®), progesterone (CRINONE®), natural progesterone, synthetic progesterone, norethindrone (AYGESTIN®) (CAMILA®), drospirenone, levonorgestrel (MIRENA®) (PLAN B®), megastrol (MEGACE®), selective estrogen receptor modulators (SERMS), tamoxifen (NOLVADEX®), raloxifene (EVISTA®), bazedoxifene.

Oral contraceptive therapeutics comprise, but are not limited to, gonadotropin-releasing hormone (GnRH) inhibitors, pituitary hormone inhibitors, estradiol, ethinyl estradiol, estrogen with progestin, estrogen without progestin, progestins, Norgestimate, etonogestrel (IMPLANON®), desogestrel, levonorgestrel (MIRENA®) (PLAN B®), norethindrone (AYGESTIN®) (CAMILA®), norethindrone acetate, ethynodiol diacetate, dienogest (NATAZIA®), drospirenone, progesterone.

Corticosteroid therapeutics comprise, but are not limited to, Class I corticosteroids, Class II corticosteroids, Class III corticosteroids, Class IV corticosteroids, Class V corticosteroids, Class VI corticosteroids, Class VII corticosteroids, Betamethasone dipropionate, Betamethasone valerate, Clobetasol propionate, Diflorasone diacetate, Halobetasol propionate, Amcinonide, Desoximetasone, Fluocinonide, Fluocinolone acetonide, Halcinonide, Mometasone furoate, Fluticasone propionate, Triamcinolone acetonide, Flurandrenolide, Desonide, Hydrocortisone, Hydrocortisone acetate, Hydrocortison acetate and pramoxine hydrochloride, Hydrocortisone butyrate, Hydrocortisone valerate, Alclometasone dipropionate, Flumethasone pivalate, hydrocortisone types, acetonides and related substances, betamethasone types, esters, halogenated esters, labile prodrug esters.

Antibiotics comprise, but are not limited to, sulfa drugs, cephalosporins, macrolides, penicillin derivatives, quinolones, tetracycline derivatives, aminoglycosides, trimethoprim (PRIMSOL®) (PROLOPRIM®) (TRIMPEX®), Quinupristin/dalfopristin, (SYNERCID®), Piperacillin/tazobactam, (ZOSYN®), Penicillin G procaine, clarithromycin, BIAXIN®), Nitrofurantoin (FURADANTIN®) (MACROBID®) (MACRODANTIN®), Ciprofloxacin (CILOXAN®) (CIPRO®), telithromycin (KETEK®), Metronidazole (FLAGYL®), levofloxacin (IQUIX®) (LEVAQUIN®) (QUIXIN®), erythromycin (ERY-TAB®), (ERYTHROCIN®), Theophylline (ELIXOPHYLLIN®), Gemifloxacin (FACTIVE®), Tetracycline (ACHROMYCIN V®), azithromycin (ZITHROMAX®), Delafloxacin (DELAFLOXACIN®), Eravacycline (ERAVACYCLINE®), moxifloxacin (AVELOX®), Dalbavancin (DALVANCE®), Amoxicillin (AMOXIL®), Fidaxomicin (DIFICID®), Tigecycline (TYGACIL®), ceftriaxone (ROCEPHIN®), Minocycline (MINOCIN®), Rifapentine (PRIFTIN®), Clindamycin (CLEOCIN®), Ceftazidime (FORTAZ®) (TAZICEF®), Oritavancin (ORBACTIV®), Norfloxacin, (NOROXIN®), Doxycycline (PERIOSTAT®) (VIBRAMYCIN®), Cefuroxime (CEFTIN®) (ZINACEF®), Tobramycin (TOBI®) (TOBREX®), Ceftibuten (CEDAX®), Gentamicin (GENOPTIC®), Cefotaxime (CLAFORAN®), Vancomycin (VANCOCIN®), Telavancin (VIBATIV®), Daptomycin (CUBICIN®), Cephalexin (KEFLEX®), Fosfomycin (MONUROL®), tedizolid (SIVEXTRO®), Aztreonam (AZACTAM®), Nafcillin (NALLPEN IN PLASTIC CONTAINER®), Phenytoin (DILANTIN®), Ertapenem (INVANZ®), Cefazolin (ANCEF®) (KEFZOL®), Isoniazid (LANIAZID®), Doripenem (DORIBAX®), rifabutin (MYCOBUTIN®), Meropenem (MERREM®), Linezolid (ZYVOX®), ofloxacin (FLOXIN OTIC®), Cefoxitin (MEFOXIN®), Oxacillin (BACTOCILL IN PLASTIC CONTAINER®), Warfarin (COUMADIN®), Neomycin (NEO-FRADIN®), Rifampin (RIFADIN®) (RIMACTANE®), Cefepime (MAXIPIME®), Digoxin (LANOXIN®).

Antiseizure therapeutics comprise, but are not limited to, broad-spectrum antiseizure drugs, focal-onset seizure drugs, generalized-onset tonic-clonic seizure drugs, epileptic spasms drugs, atonic seizures drugs, myoclonic seizures drugs, juvenile myoclonic epilepsy drugs, febrile seizure drugs, drugs for seizures due to alcohol withdrawal, anticonvulsants, Eslicarbazepine (APTIOM®), Levetiracetam (KEPPRA®), oxcarbazepine (TRILEPTAL®), Acetazolamide (DIAMOX®), Carbamazepine (TEGRETOL®), phenobarbital, Fosphenytoin (CEREBYX®), Ethosuximide (ZARONTIN®), lamotrigine (LAMICTAL®), Zonisamide (ZONEGRAN®), lacosamide (VIMPAT®), topiramate (TOPAMAX®), vigabatrin (SABRIL®), clonazepam (KLONOPIN®), pregabalin (LYRICA®), gabapentin (NEURONTIN®), felbamate (FELBATOL®), phenytoin (DILANTIN®), Ezogabine (POTIGA®), tiagabine (GABITRIL®), clobazam (ONFI®), mephobarbital, primidone, valproic acid.

A number of vaccines which can be administered in combination with bioactive vitamins include the following: Adenovirus; Anthrax, including but not limited to AVA (BIOTHRAX®); Cholera, including but not limited to Vaxchora; Diphtheria, including but not limited to DTaP (DAPTACEL®, INFANRIX®), Td (TENIVAC®, generic), DT (-generic-), Tdap (ADACEL®, BOOSTRIX®), DTaP-IPV (KINRIX®, QUADRACEL®), DTaP-HepB-IPV (PEDIARIX®), DTaP-IPV/Hib (PENTACEL®); Hepatitis A, including but not limited to HepA (HAVRIX®, VAQTA®), HepA-HepB (TWINRIX®); Hepatitis B including but not limited to HepB (ENGERIX-B®, RECOMBIVAX HB®, HEPLISAV-B®), DTaP-HepB-IPV (PEDIARIX®), HepA-HepB (TWINRIX®); Haemophilus influenzae type b (Hib) including but not limited to Hib (ACTHIB®, PEDVAXHIB®, HIBERIX®), DTaP-IPV/Hib (PENTACEL®); Human Papillomavirus (HPV) including but not limited to HPV9 (9vHPV, GARDASIL 9®); Seasonal Influenza (Flu) including but not limited to IIV* (AFLURIA®, FLUAD®, FLUBLOK®, FLUCELVAX®, FLULAVAL®, FLUARIX®, FLUVIRIN®, FLUZONE®, FLUZONE HIGH-DOSE®, FLUZONE INTRADERMAL®), LAIV (FLUMIST®); Japanese Encephalitis including but not limited to JE (IXIARO®); Measles including but not limited to MMR (M-M-R II®), MMRV (PROQUAD)®; Meningococcal including but not limited to MenACWY (MENACTRA®, MENVEO®), MenB (BEXSERO®, TRUMENBA®); Mumps including but not limited to MMR (M-M-R II®), MMRV (PROQUAD®); Pertussis including but not limited to DTaP (DAPTACEL®, INFANRIX®), Tdap (ADACEL®, BOOSTRIX®), DTaP-IPV (KINRIX®, QUADRACEL®), DTaP-HepB-IPV (PEDIARIX®), DTaP-IPV/Hib (PENTACEL®); Pneumococcal including but not limited to PCV13 (PREVNAR13®), PPSV23 (PNEUMOVAX 23®); Polio including but not limited to Polio (IPOL®), DTaP-IPV (KINRIX®, QUADRACEL®), DTaP-HepB-IPV (PEDIARIX®), DTaP-IPV/Hib (PENTACEL®); Rabies including but not limited to Rabies (IMOVAX RABIES®, RABAVERT®); Rotavirus including but not limited to RV1 (ROTARIX®), RV5 (ROTATEQ®); Rubella including but not limited to MMR (M-M-R II®), MMRV (PROQUAD®); Shingles including but not limited to ZVL (ZOSTAVAX®), RZV (SHINGRIX®); Smallpox including but not limited to Vaccinia (ACAM2000®); Tetanus including but not limited to DTaP (DAPTACEL®, INFANRIX®), Td (TENIVAC®, generic), DT (-generic-), Tdap (ADACEL®, BOOSTRIX®), DTaP-IPV (KINRIX®, QUADRACEL®), DTaP-HepB-IPV (PEDIARIX®), DTaP-IPV/Hib (PENTACEL®); Tuberculosis; Typhoid Fever including but not limited to Typhoid Oral (VIVOTIF®), Typhoid Polysaccharide (TYPHIM VI®); Varicella including but not limited to VAR (VARIVAX®), MMRV (PROQUAD®); Yellow Fever including but not limited to YF (YF-VAX®); SARS-CoV-2.

HIV therapeutics comprise, but are not limited to, antiretroviral therapy (ART), antiretrovirals, nucleoside reverse transcriptase inhibitors (NRTIs), nucleotide reverse transcriptase inhibitors (nRTIs), non-nucleoside reverse transcriptase inhibitors (NNRTIs), protease inhibitors (PIs), entry inhibitors (EIs), post-attachment inhibitors, integrase inhibitors, cyclophosphamide (CYTOXAN®), clarithromycin (BIAXIN®), Fosamprenavir (LEXIVA®), Emtricitabine (EMTRIVA®), ketoconazole (NIZORAL®), itraconazole (SPORANOX®), Elvitegravir (ELVITEGRAVIR®), Dolutegravir (DOLUTEGRAVIR®), Rilpivirine (EDURANT®), Enfuvirtide (FUZEON®), Bictegravir (BICTEGRAVIR®), Raltegravir (ISENTRESS®), didanosine (VIDEX®), Saquinavir (INVIRASE®), Atazanavir (REYATAZ®), Lamivudine (EPIVIR®), Nelfinavir (VIRACEPT®), Doravirine (DORAVIRINE®), Zidovudine (RETROVIR®), Etravirine (INTELENCE®), Tipranavir (APTIVUS®), Nevirapine (VIRAMUNE®), Ibalizumab (IBALIZUMAB®), Stavudine (ZERIT®), Maraviroc (SELZENTRY®), estradiol (ESTRADERM®) (ESTROGEL®) (VIVELLE®), ritonavir (NORVIR®), Darunavir (PREZISTA®), Tenofovir (VIREAD®), Efavirenz (SUSTIVA®), methadone (DOLOPHINE®), cisapride (PROPULSID®), warfarin (COUMADIN®), fentanyl (ACTIQ®) (DURAGESIC®) (SUBLIMAZE®), Abacavir (ZIAGEN®).

Antidepressant therapeutics comprise, but are not limited to, selective serotonin reuptake inhibitors (SSRIs), serotonin modulators (5-HT₂ blockers), serotonin-norepinephrine reuptake inhibitors, norepinephrine-dopamine reuptake inhibitors, heterocyclic antidepressants, monoamine oxidase inhibitors (MAOIs), melatonergic antidepressant, ketamine-like drugs, dextromethorphan (DELSYM®), tranylcypromine (PARNATE®), pseudoephedrine (AFRINOL®) (SUDAFED®), levomilnacipran (FETZIMA®), chlorpromazine, desvenlafaxine (PRISTIQ®), Protriptyline (VIVACTIL®), isocarboxazid (MARPLAN®), carbamazepine (TEGRETOL®), nortriptyline (AVENTYL®), clomipramine (ANAFRANIL®), vortioxetine (VORTIOXETINE®), theophylline (ELIXOPHYLLIN®), escitalopram (LEXAPRO®), Trimipramine (TRIMIPRAMINE®), desipramine (NORPRAMIN®), propranolol (INDERAL®), fluvoxamine (LUVOX®), venlafaxine (EFFEXOR XR®), Mirtazapine (REMERON®), amphetamine (ADDERALL XR 10®), paroxetine (PAXIL®), sertraline (ZOLOFT®), selegiline (ELDEPRYL®), vilazodone (VILAZODONE®), phenelzine (NARDIL®), citalopram (CELEXA®), fluoxetine (PROZAC®) (SARAFEM®), imipramine (TOFRANIL®), meperidine (DEMEROL®), metoprolol (LOPRESSOR®) (TOPROL-XL®), duloxetine (CYMBALTA®), esketamine (ESKETAMINE®), Trazodone (OLEPTRO®), Bupropion (WELLBUTRIN®) (ZYBAN®), clozapine (CLOZARIL®), ketamine (KETALAR®), warfarin (COUMADIN®), Doxepin (ZONALON®).

Cholesterol therapeutics comprise, but are not limited to, statins, bile acid sequestrants, cholestyramine, cholesterol absorption inhibitors, PCSK9 monoclonal antibodies, dietary supplements, drugs for homozygous familial hypercholesterolemia, fibrates, omega-3 fatty acids, Apo CIII inhibitor, nicotinic acid (niacin), cholesterol ester transport protein (CETP) inhibitors, Pitavastatin (LIVALO®), Rosuvastatin (CRESTOR®), Atorvastatin (LIPITOR®), cyclosporine (NEORAL®) (SANDIMMUNE®), Pravastatin (PRAVACHOL®), gemfibrozil (LOPID®), Bezafibrate, Fluvastatin (LESCOL®), Colesevelam (WELCHOL®), Simvastatin (ZOCOR®), Lomitapide (JUXTAPID®), tacrolimus (PROGRAF®), Mipomersen (KYNAIVIRO®), alirocumab (PRALUENT®), evolocumab (REPATHA®), Lovastatin (ALTOPREV®), Colestipol (COLESTID®), Metformin (GLUCOPHAGE®), Ezetimibe (ZETIA®), warfarin (COUMADIN®), digoxin (LANOXIN®), niacin (NIACOR®) (NIASPAN®).

Gout therapeutics comprise, but are not limited to, nonsteroidal anti-inflammatory drugs (NSAIDs), corticosteroids, uriosuric therapy, uriosuric agents, clarithromycin (BIAXIN®), mercaptopurine (PURINETHOL®), nitroglycerin (NITRO-DUR®), acetazolamide (DIAMOX®), azathioprine (IMURAN®), prednisolone (ORAPRED®) (PRELONE®), cyclosporine (NEORAL®) (SANDIMMUNE®), allopurinol (ZYLOPRIM®), pegloticase (KRYSTEXXA®), prednisone (RAYOS®), colchicine (COLCRYS®), probenecid, febuxostat (ULORIC®), losartan (COZAAR®), anakinra (KINERET®).

Blood pressure therapeutics comprise, but are not limited to, adrenergic modifiers, central alpha-2-agonists, postsynaptic alpha-1-blockers, peripheral-acting adrenergic blockers, angiotensin-converting enzyme (ACE) inhibitors, angiotensin II receptor blockers (ARBs), beta-blockers, calcium channel blockers, dihydropyridines, nondihydropyridines, direct renin inhibitors, direct vasoldilators, diuretics, loop diuretics, potassium-sparing diuretics, thiazide-type diuretics, Perindopril erbumine (Perindopril erbumine®), perindopril (ACEON®), Hydrochlorothiazide (MICROZIDE®), Methyclothiazide, Ethacrynic acid (EDECRIN®), Spironolactone (ALDACTONE®), chlorthalidone (THALITONE®), Chlorothiazide (DIURIL®), Trandolapril (MAVIK®), triamterene (DYRENIUM®), Candesartan (ATACAND®), Telmisartan (MICARDIS®), Propranolol (INDERAL®), Nicardipine (CARDENE®), Nisoldipine (SULAR®), Lisinopril (PRINIVIL®) (ZESTRIL®), Amlodipine (NORVASC®), carvedilol (COREG®), Furosemide (LASIX®), nifedipine (ADALAT CC®) (PROCARDIA®), Benazepril (LOTENSIN®), Irbesartan (AVAPRO®), Eprosartan (TEVETEN®), Guanfacine (TENEX®), Felodipine (PLENDIL®), Olmesartan (BENICAR®), acebutolol (SECTRAL®), metoprolol (LOPRESSOR®) (TOPROL-XL®), Azilsartan (EDARBI®), methyldopa, bisoprolol (ZEBETA®), eplerenone (INSPRA®), verapamil (CALAN®), Captopril (CAPOTEN®), amiloride (MIDAMOR®), Quinapril (ACCUPRIL®), diltiazem (CARDIZEM®) (CARTIA XT®) (DILACOR XR®), betaxolol (BETOPTIC®), Enalapril (VASOTEC®), torsemide (DEMADEX®), Valsartan (DIOVAN®), Terazosin (HYTRIN®), Aliskiren (TEKTURNA®), carteolol (OCUPRESS®), Nebivolol (BYSTOLIC®), Doxazosin (CARDURA®), minoxidil (ROGAINE®), Clonidine (CATAPRES®), Ramipril (ALTACE®), Losartan (COZAAR®), Prazosin (MINIPRESS®), atenolol (TENORMIN®), lithium (LITHOBID®), Nadolol (CORGARD®), Timolol (TIMOPTIC®), bumetanide (BUMEX®), indapamide, hydralazine (DRALZINE®) (APRESOLINE®), fosinopril, moexipril (UNIVASC®).

Osteoporosis therapeutics comprise, but are not limited to, calcium and vitamin D supplements, antiresorptive drugs, bisphosphonates, hormone replacement therapy, selective estrogen receptor modulators, activator of nuclear facor kappa-B ligand (RANKL) inhibitor, anabolic agents, parathyroid hormone (PTH) analogues, romosozumab, medroxyprogesterone (PROVERA®), cholecalciferol, Zoledronic acid (ZOMETA®), ergocalciferol (DRISDOL®), abaloparatide (TYMLOS®), rosiglitazone (AVANDIA®), teriparatide (FORTEO®), pioglitazone (ACTOS®), tetracycline (ACHROMYCIN V®), Ibandronate (BONIVA®), Alendronate (FOSAMAX®), Risedronate (ACTONEL®), Raloxifene (EVISTA®), calcitriol (ROCALTROL®), calcitonin (MIACALCIN®), denosumab (PROLIA®), heparin (PANHEPRIN®).

Antipsychotic therapeutics comprise, but are not limited to, dopamine-2 blockers, second-generation psychotics (SGAs), Diphenhydramine, Trihexyphenidyl, norepinephrine (LEVOPHED®), Brexpiprazole (REXULTI®), Paliperidone (INVEGA®), Aripiprazole (ABILIFY®), Fluphenazine, Thiothixene (NAVANE®), ziprasidone (GEODON®), Iloperidone (FANAPT®), Benztropine (COGENTIN®), risperidone (RISPERDAL®), Cariprazine (VRAYLAR®), haloperidol (HALDOL®), Lurasidone (LATUDA®), olanzapine (ZYPREXA®), Clonazepam (KLONOPIN®), quetiapine (SEROQUEL®), Lorazepam (ATIVAN®), clozapine (CLOZARIL®), Loxapine (ADASUVE®), Pimozide (ORAP®), Lithium (LITHOBID®).

Constipation therapeutics comprise, but are not limited to, osmotic laxatives, bulking agents, osmotic agents, secretory or stimulant cathartics, enemas, emollient agents, peripherally acting mu-opioid receptor antagonists (PAMORAs), magnesium sulfate, Lubiprostone (AMITIZA®), Linaclotide (LINZESS®), lidocaine (XYLOCAINE®), Lactulose (CHOLAC®), Bisacodyl (DULCOLAX®), senna, miralax, psyllium, calcium polycarbophil, methylcellulose, phenolphthalein, anthraquinones, castor oil, ducosate, mineral oil, lactulose.

Anti-Parkinson's therapeutics comprise, but are not limited to, Carbidopa/levodopa (DUOPA®) (RYTARY®) (SINEMET®) (SINEMET CR®), Amantadine (GOCOVRI®) (OSMOLEX ER®), MAO type B (MOA-B) inhibitors, anticholinergic drugs, dopamine agonists, catechol O-methyltransferase (COMT) inhibitors, oral dopamine agonists, trimethobenzamide (TIGAN®), diphenhydramine, Trihexyphenidyl Bromocriptine (PARLODEL®), Benztropine (COGENTIN®), risperidone (RISPERDAL®), pramipexole (MIRAPEX®), apomorphine (APOKYN®), amphetamine (ADDERALL XR 10®), rasagiline (AZILECT®), entacapone (COMTAN®), selegiline (ELDEPRYL®), olanzapine (ZYPREXA®), rotigotine (NEUPRO®), quetiapine (SEROQUEL®), ropinirole (REQUIP®), tolcapone (TOLCAPONE®), clozapine (CLOZARIL®), carbidopa (LODOSYN®), bisacodyl (DULCOLAX®), levodopa (LEVODOPA®).

Pain therapeutics comprise, but are not limited to, nonopioid analgesics, opioid analgesics, antidepressants, antiseizure drugs, central nervous system (CNS)-active drugs, adjuvant analgesic drugs, nonsteroidal anti-inflammatory drugs (NSAIDs), aspirin, PERCOCET®, codeine, TUZISTRA XR®, guaifenesin, hydrocodone (ZOHYDRO ER®), Choline magnesium trisalicylate, Dextroamphetamine (DEXEDRINE®), Dextromethorphan (DELSYM®), prochlorperazine (COMPRO®), diphenhydramine, Methylphenidate (CONCERTA®) (RITALIN®), norepinephrine (LEVOPHED®), Metoclopramide (REGLAN®), Mefenamic acid (PONSTEL®), Oxcarbazepine (TRILEPTAL®), Hydromorphone (DILAUDID®), Dexamethasone (OZURDEX®), Acetaminophen (TYLENOL®), Buprenorphine (BUPRENEX®), Carbamazepine (TEGRETOL®), nortriptyline (AVENTYL®), Indomethacin (INDOCIN®), lubiprostone (AMITIZA®), testosterone (DELATESTRYL®), Flurbiprofen (ANSAID®) (OCUFEN®), desipramine (NORPRAMIN®), misoprostol (CYTOTEC®), venlafaxine (EFFEXOR XR®), Hydroxyzine (VISTARIL®), pentazocine (TALWIN®), Pamidronate (AREDIA®), tamsulosin (FLOMAX®), Prednisone (RAYOS®), Ketoprofen (NEXCEDE®), pregabalin (LYRICA®), Tizanidine (ZANAFLEX®), Meperidine (DEMEROL®), gabapentin (NEURONTIN®), ziconotide (PRIALT®), Naltrexone (REVIA®), diclofenac (CATAFLAM®) (VOLTAREN®), duloxetine (CYMBALTA®), Fenoprofen (NALFON®), Ibuprofen (ADVIL®) (MOTRIN IB®), bupropion (WELLBUTRIN®) ZYBAN®), ketorolac (SPRIX®), Capsaicin (QUTENZA®), lidocaine (XYLOCAINE®), Celecoxib (CELEBREX®), Phenytoin (DILANTIN®), Modafinil (PROVIGIL®), lactulose (CHOLAC®), Oxycodone (OXYCONTIN®), Memantine (NAMENDA®), Piroxicam (FELDENE®), Methadone (DOLOPHINE®), Oxaprozin (DAYPRO®), bisacodyl (DULCOLAX®), clonidine (CATAPRES®), naloxone (EVZIO®), Tramadol (ULTRAM®), Sulindac (CLINORIL®), Naproxen (ALEVE®) (NAPROSYN®), morphine (DURAMORPH PF®) (MS CONTIN®), Fentanyl (ACTIQ®) (DURAGESIC®) (SUBLIMAZE®), Baclofen (LIORESAL®).

Oral therapeutics comprise, but are not limited to, toothpaste, dental plaque removers, halitosis suppressing treatments, dental disorder treatments, oral disorder treatments, fluoride, gingivitis treatments, baking soda, salt, lidocaine (XYLOCAINE®), saline rinses, topical gum infection agents, hydrogen peroxide.

Topical therapeutics comprise, but are not limited to, shampoos, conditioners, hair lotions, moisturizers, psoriasis treatments, immunosuppressants, emollients, salicylic acid, coal tar, anthralin, corticosteroids, vitamin D3 analogs, calcineurin inhibitors tazarotene, oral retinoids, cyclosporine, immunomodulatory agents, skin supplements and powders, hair supplements and powders, nail supplements and powder, lip balms, insecticides, keratolytics, astringents, antipruritics, sunscreens.

Wound therapeutics comprise, but are not limited to, bandages, wound healings, burn dressings, disinfectants, sterilizers, antiseptic agents, iodine solutions, NEOSPORIN®.

Nutritional uses includes, but are not limited to, vegan meat replacement products, vegan fish replacement products, food fortifications, protein powders, protein bars, nutritional supplements, enteral nutrition, medicinal foods, small and large animal medications, small and large animal feeds, veterinary treatments, infant formulas, breastfeeding alternatives, milk powders, concentrated liquids, prediluted liquids, energy drinks, patches, injections, antifungals.

Infertility therapeutics comprise, but are not limited to, sperm disorder treatments, Medroxyprogesterone (PROVERA®), Cyclophosphamide (CYTOXAN®) (LYOPHILIZED), Spironolactone (ALDACTONE®), Nitrofurantoin (FURADANTIN®) (MACROBID®) (MACRODANTIN®), Sulfasalazine (AZULFIDINE®), bicalutamide (CASODEX®), Ketoconazole (NIZORAL®), Chlorambucil (LEUKERAN®), cyproterone, Colchicine (COLCRYS®), Cimetidine (TAGAMET®), Clomiphene (CLOMID®), Estrogens, ovulatory dysfunction treatments, exogenous gonadotropins, progesterone (CRINONE®), gonadorelin, metformin (GLUCOPHAGE®), letrozole (FEMARA®), decreased ovarian reserve (DOR) treatments, donor oocytes, estradiol (ESTRADERM®) (ESTROGEL®) (VIVELLE®), tubal dysfunction and pelvic lesions treatments, abnormal cervical mucus treatments, controlled ovarian stimulation (COS) treatments, urofollitropin (BRAVELLE®) (FERTINEX®), synthetic human chorionic gonadotropin (hCG), follicle stimulating hormone (FSH), methylprednisolone (MEDROL®).

Dementia therapeutics comprise, but are not limited to, cholinesterase inhibitors, rivastigmine (EXELON®), benztropine (COGENTIN®), propranolol (INDERAL®), galantamine (RAZADYNE®), memantine (NAMENDA®), donepezil (ARICEPT®).

Macular degeneration therapeutics comprise, but are not limited to, lutein, zeaxanthin, beta-carotene, antivascular endothelial growth factor (anti-VEGF) drugs, corticosteroids, triamcinolone (KENALOG®), bevacizumab (AVASTIN®), aflibercept (AFLIBERCEPT®), ranibizumab (LUCENTIS®).

Fibromyalgia therapeutics comprise, but are not limited to, tricyclic antidepressants, nonopioid analgesics, cyclobenzaprine (AMRIX®), amitriptyline, acetaminophen (TYLENOL®), milnacipran (SAVELLA®), bupivacaine (MARCAINE®), Pregabalin (LYRICA®), duloxetine (CYMBALTA®), trazodone (OLEPTRO®), lidocaine (XYLOCAINE®), doxepin (ZONALON®).

### Acetaminophen+Vitamers

The vitamers have anti-pain benefits to them so combining them with prescribed (opioids) and OTC medications would amplify the benefits of each. Please note in the reference below cyanocobalamin was used with benefit so the better vitamer form of methylcobalamin and adenosylcobalamin would be a more enhancing choice. See Hosseinzadeh H, et al. Ibuprofen may also be used in lieu of, or in addition to, acetaminophen.

### Acetaminophen+Vitamers+N-Acetylcysteine (NAC)

When acetaminophen is used to treat chronic pain and taken on a daily basis, there is an increased risk for liver damage. NAC (600 mg) is already approved and prescribed for acetaminophen overdoses. Combining them in one pill would be of great protective benefit for long term use. Supportive references include Heard K J. Acetylcysteine for acetaminophen poisoning. N Engl J Med. 2008; 359(3):285-292; Hosseinzadeh H, et al, “Anti-Nociceptive and Anti-Inflammatory Effects of Cyanocobalamin (Vitamin B12) Against Acute and Chronic Pain and Inflammation in Mice,” Arzneimittelforschung, 2012; 62(7):324-9 (“In conclusion our work presents some experimental evidence supporting the administration of cyanocobalamin in controlling acute and chronic neuropathic pain. Cyanocobalamin may have anti-inflammatory effect”); Mauro, G. L., et al, “Vitamin B12 in Low Back Pain: A Randomised, Double-Blind, Placebo-Controlled Study,” Eur Rev Med Pharmacol Sci, 2000; 4(3):53-8 (“The efficacy and safety of parenteral Vitamin B12 in alleviating low back pain and related disability and in decreasing the consumption of paracetamol[acetaminophen] was confirmed in patients with no signs of nutritional deficiency.”).

### Intravenous Formulation of Vitamers by Themselves

The vitamers described herein may be administered as intravenous formulations. See, e.g., Flynn, M. A., Irvin, W., Krause, G., “The Effect of Folate and Cobalamin on Osteoarthritis Hands,” J Am Coll Nutr, 1994; 13(4):351-356 (“The limited number of subjects in this study demonstrates that ingestion of a prescribed cobalamin-folate supplement and acetaminophen as needed resulted in positive outcomes.”).

### Intramuscular Injection Formulation by Itself

The intravenous formulation as well as intramuscular formulation would need to be in liquid form at the same doses mentioned previously in the listings. Another way to lengthen shelf-life would be to prepare it just prior to use, as many other parenteral medications are, by having just the powder in the vial and then reconstitute it in the bottle by injecting sterile saline. These injectable and intravenous forms would make it available for routine and emergency use for surgery, emergency conditions or for very low blood levels just as they are now.

### Target Cancers

The cancer can be a solid tumor. The cancer can be a hematological cancer. In certain embodiments, the cancer is a solid tumor selected from the group consisting of squamous cell carcinoma, non-squamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST).

In particular embodiments, the cancer is a solid tumor selected from non-small cell lung cancer (NSCLC), hepatocellular carcinoma, melanoma, ovarian cancer, breast cancer, pancreatic cancer, renal cell carcinoma, or colorectal cancer. The cancer can be non-small cell lung cancer (NSCLC). The cancer can be hepatocellular carcinoma. The cancer can be melanoma. The cancer can be ovarian cancer. The cancer can be breast cancer. The cancer can be pancreatic cancer. The cancer can be renal cell carcinoma. The cancer can be colorectal cancer.

Provided herein are methods of treating NSCLC by administering a therapeutically effective amount of a combination described herein where the combination includes a combination of vitamers and bioactive compounds. In some embodiments, the NSCLC is Stage IIA or Stage IIB. The NSCLC can be a Stage IIIA or Stage IIIB cancer. The NSCLC can be a Stage IV cancer. Staging of cancers as described herein is described by the American Joint Committee on Cancer TNM classification of malignant tumors cancer staging notation as is well understood in the art. Those of skill in the art will readily understand other staging classification systems are available and applicable to the methods described herein. In certain embodiments, the method is a method of treating Stage IIIA or IIIB NSCLC by administering a combination described herein that includes a combination of Vitamers and bioactive compounds and an Inhibitor Antibody.

Still further provided herein are methods of treating melanoma by administering a therapeutically effective amount of a combination described herein where the combination includes a combination of vitamers and bioactive compounds. In some embodiments the melanoma is a Stage IIA, IIB, or IIC cancer. In another embodiment, the melanoma is a Stage IIIA, Stage IIIB, or Stage IIIC cancer. In still another embodiment, the melanoma is a Stage IV cancer. In one aspect the method is a method of treating Stage II (e.g., Stage IIA, IIB, or IIC) melanoma by administering a therapeutically effective amount of a combination described herein where the combination includes a combination of vitamers and bioactive compounds.

Also provided herein are methods of treating breast cancer by administering a therapeutically effective amount of a combination described herein where the combination includes a combination of vitamers and bioactive compounds. The breast cancer can be HER2 negative breast cancer. The breast cancer can be a HER2 positive breast cancer. The breast cancer can be triple-negative breast cancer. In some embodiments the breast cancer is a Stage IA or Stage IB cancer. In another embodiment, the breast cancer is a Stage IIA or Stage IIB cancer. In still another embodiment, the breast cancer is a Stage IIIA, Stage IIIB, or Stage IIIC cancer. In yet another embodiment, the breast cancer is a Stage IV cancer.

In other embodiments, the cancer is a hematological cancer selected from lymphoma, Non-Hodgkin's lymphoma (NHL), Hodgkin's Lymphoma, Reed-Sternberg disease, multiple myeloma (MM), acute myelogenous leukemia (AML), chronic myelogenous leukemia (CML), acute lymphocytic leukemia, (ALL), or chronic lymphocytic leukemia (CLL). In certain embodiments, the cancer is Hodgkin's Lymphoma or Reed-Sternberg disease.

The combinations described herein can be administered to a cancer patient at any time following diagnosis. For example, the cancer patient can be treatment naïve (e.g., has not received a cancer therapy for the diagnosed cancer). The cancer patient can be treatment naïve for one cancer but can be diagnosed with one or more other cancers resulting from, for example, metastasis or malignancy. The cancer patient can be immune checkpoint naïve for one or more cancers. The cancer patient can have a cancer that is refractory. In certain embodiments, the combinations described herein are administered as a first line therapy (e.g., the first therapy administered to a treatment naïve cancer patient) to a patient in need thereof.

However, cancer morbidity and mortality is often associated with ineffective therapy or a cancer gaining resistant to or becoming refractory to one or more cancer therapies. The combinations described herein can, therefore, be administered to patients in need thereof as a second, third, fourth, fifth, sixth, or more line of treatment. The combinations described herein can be administered to a cancer patient who has been treated with at least one anti-cancer therapy or anti-cancer agent. In certain embodiments the patient has received at least one anti-cancer therapy including, for example, chemotherapy, radiotherapy, surgery, targeted therapy, immunotherapy, or a combination thereof. The patient can have a cancer that is resistant/refractory to treatment with at least one anti-cancer agent.

The methods of treating cancers herein include treating subjects who have been treated with an immune checkpoint inhibitor and have experienced no response to treatment, or a partial response, or stable disease, but then develop resistance to treatment with progression of disease or who have experienced a complete response to treatment, but then develop resistance to treatment with progression of disease (as defined by RECIST or other criteria). Resistance is defined as disease progression during treatment or a lack of response to treatment. Such ICI treatment failures can be treated with an ICI in combination with a combination of vitamers and bioactive compounds.

The methods of treatment and therapies described herein also provide for methods for increasing or otherwise prolonging time to disease progression of certain stages (including advanced stages of cancer such as Stage III and IV cancer described herein). Time to disease progression can be prolonged in a patient by administering a therapeutically effective amount of a combination described herein. For example, the combination includes a combination of vitamers plus a bioactive compound described herein. In some embodiments, the increase is a comparison between the time to disease progression without treatment and with treatment with a combination described herein. In some embodiments, the methods described herein prolong the time to disease progression by at least 1 week, 2 weeks, 3 weeks, 4 weeks, 1 month, 2 months, 3 months, 4 months, 5 months, 6 months, 7 months, 8 months, 9 months, 10 months, 11 months, 1 year, or more, including values therein.

The methods of treatments and therapies described herein also provide for methods for increasing or otherwise prolonging survival (including overall survival) of patients diagnosed with cancer as described herein. Patient survival can be prolonged by administering a therapeutically effective amount of a combination described herein. For example, the combination includes a combination of vitamers, plus a bioactive compound described herein. In some embodiments, the increase is a comparison between the survival without treatment and with treatment with a combination as described herein. In some embodiments, the methods described herein prolong survival by at least 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, or more, including values therein.

The methods of treatments and therapies described herein also provide for methods for increasing progression-free survival of patients diagnosed with cancer or other diseases as described herein. Patient progression-free survival can be prolonged by administering a therapeutically effective amount of a combination described herein. For example, the combination includes a combination of vitamers, plus a bioactive compound described herein. In some embodiments, the increase is a comparison between the progression-free survival without treatment and with treatment with a combination as described herein. In some embodiments, the methods described herein increase progression-free survival by at least 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, or more, including values therein.

The combinations described herein can also enhance antibody-dependent cell-mediated cytotoxicity in a cancer patient upon administration of a combination as described herein.

Exemplary anti-cancer agents include but are not limited to: ABRAXANE; abiraterone; ace-11; aclarubicin; acivicin; acodazole hydrochloride; acronine; actinomycin; acylfulvene; adecypenol; adozelesin; adriamycin; aldesleukin; all trans-retinoic acid (ATRA); altretamine; ambamustine; ambomycin; ametantrone acetate; amidox; amifostine; aminoglutethimide; aminolevulinic acid; amrubicin; amsacrine; anagrelide; anastrozole; andrographolide; antarelix; anthramycin; aphidicolin glycinate; apurinic acid; ara-CDP-DL-PTBA; arginine deaminase; ARRY-162; ARRY-300; ARRY-142266; AS703026; asparaginase; asperlin; asulacrine; atamestane; atrimustine; axinastatin 1; axinastatin 2; axinastatin 3; azasetron; azatoxin; azatyrosine; azacitidine; AZD8330; azetepa; azotomycin; balanol; batimastat; BAY 11-7082; BAY 43-9006; BAY 869766; bendamustine; benzochlorins; benzodepa; benzoylstaurosporine; beta-alethine; betaclamycin B; betulinic acid; b-FGF inhibitor; bicalutamide; bisantrene; bisaziridinylspermine; bisnafide; bisnafide dimesylate; bistratene A; bisantrene hydrochloride; bleomycin; bleomycin sulfate; busulfan; bizelesin; breflate; bortezomib; brequinar sodium; bropirimine; budotitane; buthionine sulfoximine; bryostatin; cactinomycin; calusterone; calcipotriol; calphostin C; camptothecin derivatives; capecitabine; carboxamide-amino-triazole; carboxyamidotriazole; CaRest M3; CARN 700; caracemide; carbetimer; carboplatin; carmustine; carubicin hydrochloride; carzelesin; castanospermine; cecropin B; cedefingol; celecoxib; cetrorelix; chlorins; chloroquinoxaline sulfonamide; cicaprost; chlorambucil; Chlorofusin; cirolemycin; cisplatin; CI-1040; cis-porphyrin; cladribine; clomifene analogues; clotrimazole; collismycin A; collismycin B; combretastatin A4; combretastatin analogue; conagenin; crambescidin 816; crisnatol; crisnatol mesylate; cryptophycin 8; cryptophycin A derivatives; curacin A; cyclopentanthraquinones; cycloplatam; cypemycin; cyclophosphamide; cytarabine; cytarabine ocfosfate; cytolytic factor; cytostatin; dacarbazine; dactinomycin; daunorubicin; daunorubicin hydrochloride; decarbazine; dacliximab; dasatinib; decitabine; dehydrodidemnin B; deslorelin; dexamethasone; dexifosfamide; dexrazoxane; dexverapamil; dexormaplatin; dezaguanine; dezaguanine mesylate; diaziquone; didemnin B; didox; diethylnorspermine; dihydro 5 azacytidine; dihydrotaxol; 9-dioxamycin; diphenyl spiromustine; docosanol; dolasetron; docetaxel; doxorubicin; doxorubicin hydrochloride; doxifluridine; droloxifene; droloxifene citrate; dromostanolone propionate; dronabinol; duazomycin; duocarmycin SA; ebselen; ecomustine; edelfosine; edrecolomab; edatrexate; eflornithine hydrochloride; eflornithine; elemene; emitefur; elsamitrucin; enloplatin; enpromate; epipropidine; epirubicin; epirubicin hydrochloride; epristeride; erbulozole; eribulin;esorubicin hydrochloride; estramustine; estramustine phosphate sodium; etanidazole; etoposide; etoposide phosphate; etoprine; exemestane; fadrozole; fadrozole hydrochloride; fazarabine; fenretinide; filgrastim; finasteride; flavopiridol; flezelastine; fluasterone; floxuridine; fludarabine phosphate; fludarabine; fluorodaunorubicin hydrochloride; forfenimex; formestane; fluorouracil; floxouridine; flurocitabine; fosquidone; fostriecin sodium; fostriecin; fotemustine; gadolinium texaphyrin; gallium nitrate; galocitabine; ganirelix; gelatinase inhibitors; gemcitabine; geldanamycin; gossyphol; GDC-0973; GSK1120212/trametinib; herceptin; hydroxyurea; hepsulfam; heregulin; hexamethylene bisacetamide; hypericin; ibandronic acid; ibrutinib; idarubicin; idarubicin hydrochloride; ifosfamide; canfosfamide; ilmofosine; iproplatin; idoxifene; idramantone; ilmofosine; ilomastat; imidazoacridones; imatinib (e.g., GLEEVEC); imiquimod; iobenguane; iododoxorubicin; ipomeanol; irinotecan; irinotecan hydrochloride; irsogladine; isobengazole; isohomohalicondrin B; itasetron; iimofosine; interleukin I1 (including recombinant interleukin IL-2; or r1L.sub.2); interferon alfa-2a; interferon alfa-2b; interferon alfa-n1; interferon alfa-n3; interferon beta-1a; interferon gamma-1b; jasplakinolide; kahalalide F; lamellarin N triacetate; lanreotide; leinamycin; lenograstim; lentinan sulfate; leptolstatin; letrozole; leuprorelin; levamisole; liarozole; lissoclinamide 7; lobaplatin; lombricine; lometrexol; lonidamine; losoxantrone; lovastatin; loxoribine; lurtotecan; lutetium texaphyrin; lysofylline; lanreotide acetate; lapatinib; letrozole; leucovorin; leuprolide acetate; liarozole hydrochloride; lometrexol sodium; lomustine; lenalidomide; lenvatinib; losoxantrone hydrochloride; LY294002; pomalidomide; maitansine; mannostatin A; marimastat; masoprocol; maspin; matrilysin inhibitors; menogaril; merbarone; meterelin; methioninase; metoclopramide; MIF inhibitor; mifepristone; miltefosine; mirimostim; mitoguazone; mitolactol; mitonafide; mitoxantrone; mofarotene; molgramostim; mopidamol; mycaperoxide B; myriaporone; maytansine; mechlorethamine hydrochloride; megestrol acetate; melengestrol acetate; melphalan; mercaptopurine; metoprine; meturedepa; mitindomide; mitocarcin; mitocromin; mitogillin; mitomalcin; mitomycin; mitosper; mitotane; mitoxantrone hydrochloride; mycophenolic acid; nafarelin; nagrestip; napavin; naphterpin; nartograstim; nedaplatin; nemorubicin; neridronic acid; nilutamide; nisamycin; nitric oxide modulators; nitroxide antioxidant; nitrullyn; nocodazole; nogalamycin; oblimersen (GENASENSE); octreotide; okicenone; oligonucleotides; onapristone; ondansetron; ondansetron; oracin; oral cytokine inducer; ormaplatin; oxisuran; oxaloplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; porfiromycin; prednisone; prostaglandin J2; pyrazoloacridine; paclitaxel; PD035901; PD184352; PD318026; PD98059; peliomycin; pentamustine; peplomycin sulfate; PKC412; pipobroman; piposulfan; piroxantrone hydrochloride; plicamycin; plomestane; podophyllotoxin; polyphenol E; porfimer sodium; porfiromycin; prednimustine; procarbazine; procarbazine hydrochloride; puromycin; puromycin hydrochloride; pyrazofurin; raltitrexed; ramosetron; retelliptine demethylated; rhizoxin; rituximab; RII retinamide; rogletimide; rohitukine; romurtide; roquinimex; rubiginone B1; ruboxyl; riboprine; romidepsin; safingol; safingol hydrochloride; saintopin; sarcophytol A; sargramostim; semustine; sizofiran; sobuzoxane; sodium borocaptate; sodium phenylacetate; solverol; sonermin; sorafenib; sunitinib; sparfosic acid; spicamycin D; spiromustine; splenopentin; spongistatin 1; Spongistatin 2; Spongistatin 3; Spongistatin 4; Spongistatin 5; Spongistatin 6; Spongistatin 7; Spongistatin 8; and Spongistatin 9; squalamine; stipiamide; stromelysin inhibitors; sulfinosine; suradista; suramin; swainsonine; SB239063; selumetinib/AZD6244; simtrazene; SP600125; sparfosate sodium; sparsomycin; spirogermanium hydrochloride; spiroplatin; streptonigrin; streptozocin; sulofenur; tallimustine; tamoxifen methiodide; tauromustine; tazarotene; tecogalan sodium; tegafur; tellurapyrylium; temoporfin; temozolomide; teniposide; tetrachlorodecaoxide; tetrazomine; thaliblastine; thiocoraline; thrombopoietin; thymalfasin; thymopoietin receptor agonist; thymotrinan; tirapazamine; titanocene bichloride; topsentin; toremifene; tretinoin; triacetyluridine; triciribine; trimetrexate; triptorelin; tropisetron; turosteride; tyrphostins; talisomycin; TAK-733; taxotere; tegafur; teloxantrone hydrochloride; teroxirone; testolactone; thiamiprine; thioguanine; thiotepa; tiazofurin; tirapazamine; toremifene citrate; trastuzumab; trestolone acetate; triciribine phosphate; trimetrexate; trimetrexate glucuronate; triptorelin; tubulozole hydrochloride; tumor necrosis factor-related apoptosis-inducing ligand (TRAIL); UBC inhibitors; ubenimex; U0126; uracil mustard; uredepa; vapreotide; variolin B; velaresol; veramine; verteporfin; vinorelbine; vinxaltine; vitaxin; vinblastine; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; wortmannin; XL518; zanoterone; zeniplatin; zilascorb; zinostatin stimalamer; zinostatin; and zorubicin hydrochloride.

Other exemplary anti-cancer agents include Erbulozole (e.g., R-55104); Dolastatin 10 (e.g., DLS-10 and NSC-376128); Mivobulin isethionate (e.g., CI-980); NSC-639829; Discodermolide (e.g., NVP-XX-A-296); ABT-751 (Abbott; e.g., E-7010); Altorhyrtin A; Altorhyrtin C); Cemadotin hydrochloride (e.g., LU-103793 and NSC-D-669356); Epothilone A; Epothilone B; Epothilone C; Epothilone D; Epothilone E; Epothilone F; Epothilone B N-oxide; Epothilone A N-oxide; 16-aza-epothilone B; 21-aminoepothilone B; 21-hydroxyepothilone D; 26-fluoroepothilone; Auristatin PE (e.g., NSC-654663); Soblidotin (e.g., TZT-1027); LS-4559-P (Pharmacia; e.g., LS-4577); LS-4578 (Pharmacia; e.g., LS-477-P); LS-4477 (Pharmacia); LS-4559 (Pharmacia); RPR-112378 (Aventis); DZ-3358 (Daiichi); FR-182877 (Fujisawa; e.g., WS-9265B); GS-164 (Takeda); GS-198 (Takeda); KAR-2 (Hungarian Academy of Sciences); BSF-223651 (BASF; e.g., ILX-651 and LU-223651); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (e.g., LY-355703); AC-7739 (Ajinomoto; e.g., AVE-8063A and CS-39.HC1); AC-7700 (Ajinomoto; e.g., AVE-8062; AVE-8062A; CS-39-L-Ser.HC1; and RPR-258062A); Vitilevuamide; Tubulysin A; Canadensol; CA-170 (Curis, Inc.); Centaureidin (e.g., NSC-106969); T-138067 (Tularik; e.g., T-67; TL-138067 and TI-138067); COBRA-1 (Parker Hughes Institute; e.g., DDE-261 and WHI-261); H10 (Kansas State University); H16 (Kansas State University); Oncocidin A1 (e.g., BTO-956 and DIME); DDE-313 (Parker Hughes Institute); Fijianolide B; Laulimalide; SPA-2 (Parker Hughes Institute); SPA-1 (Parker Hughes Institute; e.g., SPIKET-P); 3-IAABU (Cytoskeleton/Mt. Sinai School of Medicine; e.g., MF-569); Narcosine (e.g., NSC-5366); Nascapine; D-24851 (Asta Medica); A-105972 (Abbott); Hemiasterlin; 3-BAABU (Cytoskeleton/Mt. Sinai School of Medicine; e.g., MF-191); TMPN (Arizona State University); Vanadocene acetylacetonate; T-138026 (Tularik); Monsatrol; lnanocine (e.g., NSC-698666); 3-IAABE (Cytoskeleton/Mt. Sinai School of Medicine); A-204197 (Abbott); T-607 (Tuiarik; e.g., T-900607); RPR-115781 (Aventis); Eleutherobins (e.g., Desmethyleleutherobin; Desaetyleleutherobin; lsoeleutherobin A; and Z-Eleutherobin); Caribaeoside; Caribaeolin; Halichondrin B; D-64131 (Asta Medica); D-68144 (Asta Medica); Diazonamide A; A-293620 (Abbott); NPI-2350 (Nereus); Taccalonolide A; TUB-245 (Aventis); A-259754 (Abbott); Diozostatin; (-)-Phenylahistin (e.g., NSCL-96F037); D-62638 (Asta Medica); D-62636 (Asta Medica); Myoseverin B; D-43411 (Zentaris; e.g., D-81862); A-289099 (Abbott); A-318315 (Abbott); HTI-286 (e.g., SPA-110; trifluoroacetate salt) (Wyeth); D-82317 (Zentaris); D-82318 (Zentaris); SC-12983 (NCI); Resverastatin phosphate sodium; BPR-OY-007 (National Health Research Institutes); and SSR-250411 (Sanofi)); goserelin; leuprolide; triptolide; homoharringtonine; topotecan; itraconazole; deoxyadenosine; sertraline; pitavastatin; clofazimine; 5-nonyloxytryptamine; vemurafenib; dabrafenib; gefitinib (IRESSA); erlotinib (TARCEVA); cetuximab (ERBITUX); lapatinib (TYKERB); panitumumab (VECTIBIX); vandetanib (CAPRELSA); afatinib/BIBW2992; CI-1033/canertinib; neratinib/HKI-272; CP-724714; TAK-285; AST-1306; ARRY334543; ARRY-380; AG-1478; dacomitinib/PF299804; OSI-420/desmethyl erlotinib; AZD8931; AEE726; pelitinib/EKB-569; CUDC-101; WZ8040; WZ4002; WZ3146; AG-490; XL647; PD153035; 5-azathioprine; 5-aza-2′-deoxycytidine; 17-N-Allylamino-17-Demethoxygeldanamycin (17-AAG); 20-epi-1,25 dihydroxyvitamin D3; 5 ethynyluracil; and BMS-599626.

### Synergism

Vitamers as described herein can be provided in amounts that are synergistic with the amount of bioactive compound. The term synergistic refers to a combination described herein (e.g., a vitamer, plus a bioactive compound—including co-administration with another active agent such as an anti-cancer agent described herein) or a combination of regimens such as those described herein that is more effective than the additive effects of each individual therapy or regimen.

A synergistic effect of a combination described herein can permit the use of lower dosages of one or more of the components of the combination (e.g., a vitamer or a bioactive compound). A synergistic effect can permit less frequent administration of at least one of the administered therapies (e.g., a vitamer or a bioactive compound) to a subject with a disease, disorder, or condition described herein. Such lower dosages and reduced frequency of administration can reduce the toxicity associated with the administration of at least one of the therapies (e.g., a vitamer or a bioactive compound) to a subject without reducing the efficacy of the treatment. A synergistic effect as described herein avoid or reduce adverse or unwanted side effects associated with the use of any therapy.

### Pharmaceutical Compositions

Combinations described herein can be provided as a pharmaceutical composition suitable for administration via any route to a patient described herein including but not limited to: oral, mucosal (e.g., nasal, inhalation, pulmonary, sublingual, vaginal, buccal, or rectal), parenteral (e.g., subcutaneous, intravenous, bolus injection, intramuscular, or intra-arterial), topical (e.g., eye drops or other ophthalmic preparations), transdermal or transcutaneous administration to a patient.

The dosage required will depend on the route of administration, the nature of the formulation, the nature of the patient's illness, the patient's size, weight, surface area, age, and sex, other drugs being administered, and the judgment of the attending clinicians. Suitable dosages of bioactive compounds used in combination with bioactive vitamins are in the range of 0.001-1,000 mg/kg. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10- or more fold). Encapsulation of the compounds in a suitable delivery vehicle (e.g., polymeric microparticles or implantable devices) may increase the efficiency of delivery.

Exemplary of dosage forms include: tablets; caplets; capsules (e.g., gelatin capsules); cachets; lozenges; suppositories; powders; gels; liquid dosage forms suitable for parenteral administration to a patient; and sterile solids (e.g., crystalline or amorphous solids) that can be reconstituted to provide liquid dosage forms suitable for parenteral administration to a patient.

Pharmaceutical compositions and dosage forms described herein typically include one or more excipients. Suitable excipients are well known to those skilled in the art of pharmacy. Whether a particular excipient is suitable for incorporation into a pharmaceutical composition or dosage form depends on a variety of factors such as, for example, the intended route of administration to the patient. Pharmaceutical compositions described herein can include other agents such as stabilizers, lubricants, buffers, and disintegrants that can reduce the rate by which an active ingredient can decompose in a particular formulation.

Pharmaceutical compositions described herein can in certain instances include additional active agents other than those in the combinations described herein (e.g., an anti-cancer agent such as those described herein) in an amount provided herein.

### Kits

The combinations and pharmaceutical compositions described herein can be provided as part of a kit. Such kits can, for example, improve patient compliance or improve the accuracy or ease of preparation for administering the combination. The kit includes a combination of vitamers and bioactive compounds where the compound is supplied in a formulation as described herein.

Kits of the invention can include the combinations described herein having the same or different formulation. Each component of a combination described herein in a kit can be supplied in a separate, individual container. Alternatively, or additionally, components of the combinations described herein can be supplied in a single container. In such embodiments, the container can be a container that is ready for administration to a patient in need thereof, such as for example, an IV bag, ampoule, or a syringe. In one embodiment, the combination of Vitamers and bioactive compounds in the kit is formulated for oral administration (e.g., a tablet, capsule, or sachet).

The contents of kits described herein can be provided in sterile form. The kit and its contents can be provided in a form that is ready for administration to the subject in need. In such embodiments, the components of the combination of the kit are supplied as a formulation and optionally in an administration device such that administration requires little to no further action by the user. Where kits include administration devices, such devices include devices known and understood by those skilled in the art for routes of administration described herein, such as but not limited to, syringes, pumps, bags, cups, inhalers, droppers, patches, creams, or injectors.

### Methods

The combinations, pharmaceutical compositions, and kits described herein are useful for treating diseases, disorders, or alleviating or eliminating the symptoms of diseases and disorders such as, and in one non-limiting example, cancer. Other indications include diabetes, gastric acidity, hormone replacement effects, oral contraception effects, corticosteroid effects, antibiotic effects, seizures, tumor vaccine effects, HIV, antidepressant effects, high cholesterol, gout, high blood pressure, osteoporosis, psychosis, constipation, Parkinson's disease, pain, oral therapeutic effects, topical therapeutic effects, wound healing, nutrition, infertility, dementia, macular degeneration, fibromyalgia, and any combination thereof.

It is to be understood that the methods described herein pertain to administration of combinations and pharmaceutical compositions described herein, and such combinations and pharmaceutical compositions can be provided in the form of a kit as described herein. Provided herein are methods of treatment by administering a therapeutically effective amount of a combination described herein to a patient in need thereof. Also provided herein are methods of managing treatments or therapies by administering therapeutically effective amount of a combination described herein to a patient in need thereof.

### Combination Administration

The combinations described herein can include administration of each therapy (e.g., a combination of vitamers, plus a bioactive compound), where the administration is performed simultaneously or sequentially (in either order). In one embodiment, the combination of vitamers, plus a bioactive compound are administered simultaneously (e.g., within at least 1 to 5 min of each other). In another embodiment, the combination of vitamers, plus a bioactive compound are administered sequentially (e.g., within at least 10 min, 15 min, 30 min, 1 h, 2 h, 5 h, 10 h, 12 h, 1 day, 2 days, 5 days, 7 days, 14 days, or 21 days of each other).

The combinations (compositions) described herein comprise a compound, including vitamers and bioactive compounds, which can be administered, for example, once a day (QD), twice daily (BID), once a week (QW), twice weekly (BIW), three times a week (TIW), or monthly (QM) regularly on a continuous base or intermittent base such as BIW for 3 months then resume a month later. For example, the combination of vitamers and bioactive compounds can be administered BID. The combination of vitamers and bioactive compounds can be administered TIW. In certain embodiments, the combination of Vitamers and bioactive compounds is administered 2 to 3 times a week. In another embodiment, the combination of vitamers and bioactive compounds is administered QD. The compound can be administered QD for about: 1 day to about 7 days, 1 day to about 14 days, 1 day to about 21 days, 1 day to about 28 days, or daily until disease progression or unacceptable toxicity. The administration of a combination of vitamers and bioactive compounds can, in part, depend upon the tolerance of the patient where greater tolerance can allow greater or more frequent administration. Alternatively, where a patient shows poor tolerance to a combination of vitamers and bioactive compounds, a less amount of the compound or a less frequent dosing can be performed. Compounds of vitamers and bioactive compounds can be administered in any regimen as described herein.

For example, a bioactive compound in combination with vitamers can be administered at an amount of about: 1 μg, 2 μg, 3 μg, 4 μg, 5 μg, 10 μg, 15 μg, 20 μg, 25 μg, 30 μg, 35 μg, 40 μg, 45 μg, 50 μg, 60 μg, 70 μg, 80 μg, 85 μg, 90 μg, 100 μg, 125 μg, 150 μg, 175 μg, 200 μg, 300 μg, 400 μg, 500 μg, 600 μg, 700 μg, 800 μg, 900 μg, 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QD. For example, a bioactive compound in combination with vitamers can be administered at an amount of about: 1 μg, 2 μg, 3 μg, 4 μg, 5 μg, 10 μg, 15 μg, 20 μg, 25 μg, 30 μg, 35 μg, 40 μg, 45 μg, 50 μg, 60 μg, 70 μg, 80 μg, 85 μg, 90 μg, 100 μg, 125 μg, 150 μg, 175 μg, 200 μg, 300 μg, 400 μg, 500 μg, 600 μg, 700 μg, 800 μg, 900 μg, 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, BIW. For example, a bioactive compound in combination with vitamers can be administered at an amount of about: 1 μg, 2 μg, 3 μg, 4 μg, 5 μg, 10 μg, 15 μg, 20 μg, 25 μg, 30 μg, 35 μg, 40 μg, 45 μg, 50 μg, 60 μg, 70 μg, 80 μg, 85 μg, 90 μg, 100 μg, 125 μg, 150 μg, 175 μg, 200 μg, 300 μg, 400 μg, 500 μg, 600 μg, 700 μg, 800 μg, 900 μg, 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, TIW. For example, a bioactive compound in combination with vitamers can be administered at an amount of about: 1 μg, 2 μg, 3 μg, 4 μg, 5 μg, 10 μg, 15 μg, 20 μg, 25 μg, 30 μg, 35 μg, 40 μg, 45 μg, 50 μg, 60 μg, 70 μg, 80 μg, 85 μg, 90 μg, 100 μg, 125 μg, 150 μg, 175 μg, 200 μg, 300 μg, 400 μg, 500 μg, 600 μg, 700 μg, 800 μg, 900 μg, 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QW. For example, a composition, comprising vitamers and bioactive compounds, can be administered at an amount of about: 1 μg, 2 μg, 3 μg, 4 μg, 5 μg, 15 μg, 20 μg, 25 μg, 30 μg, 35 μg, 40 μg, 45 μg, 50 μg, 60 μg, 70 μg, 80 μg, 85 μg, 90 μg, 100 μg, 125 μg 150 μg, 175 μg, 200 μg, 300 μg, 400 μg, 500 μg, 600 μg, 700 μg, 800 μg, 900 μg, 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, Q2W. For example, a composition, comprising vitamers and bioactive compounds, can be administered at an amount of about 5 mg or about 10 mg, QD. For example, a combination of vitamers and bioactive compounds can be administered at an amount of about 5 mg or about 10 mg, BIW. For example, a combination of vitamers and bioactive compounds can be administered at an amount of about 5 mg or about 10 mg, TIW. For example, a combination of vitamers and bioactive compounds can be administered at an amount of about 5 mg or about 10 mg, QW. For example, a combination of vitamers and bioactive compounds can be administered at an amount of about 5 mg or about 10 mg, Q2W. Administration of a combination of vitamers and bioactive compounds can be continuous. Administration of a combination of vitamers and bioactive compounds can be intermittent.

For example, a composition, comprising vitamers and bioactive compounds, can be administered at an amount of about: 1 μg, to 200 mg, 10 μg to 100 mg, 100 μg to 10 mg, 1000 μg to 2 mg, QD. For example, a composition, comprising vitamers and bioactive compounds, can be administered at an amount of about: 1 μg, to 200 mg, 10 μg to 100 mg, 100 μg to 10 mg, 1000 μg to 2 mg, BIW. For example, a composition, comprising vitamers and bioactive compounds, can be administered at an amount of about: 1 μg, to 200 mg, 10 μg to 100 mg, 100 μg to 10 mg, 1000 μg to 2 mg, TIW. For example, a composition, comprising vitamers and bioactive compounds, can be administered at an amount of about: 1 μg, to 200 mg, 10 μg to 100 mg, 100 μg to 10 mg, 1000 μg to 2 mg, QW. For example, a composition, comprising vitamers and bioactive compounds, can be administered at an amount of about: 1 μg to 200 mg, 10 μg to 100 mg, 100 μg to 10 mg, 1000 μg to 2 mg, Q2W. Administration of a combination of vitamers and bioactive compounds can be continuous. Administration of a combination of vitamers and bioactive compounds can be intermittent.

For example, a composition, comprising vitamers and bioactive compounds, can be administered at an amount of about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QD. For example, a composition, comprising vitamers and bioactive compounds, can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, BIW. For example, a composition, comprising vitamers and bioactive compounds, can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, TIW. For example, a composition, comprising vitamers and bioactive compounds, can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QW. For example, a composition, comprising vitamers and bioactive compounds, can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, Q2W. In one example, a combination of vitamers and bioactive compounds can be administered at an amount of about 15 mg/kg to about 75 mg/kg, QD. In another example, a combination of vitamers and bioactive compounds can be administered at an amount of about 20 mg/kg to about 50 mg/kg. In still another example, a combination of vitamers and bioactive compounds can be administered at an amount of about 0.001 mg/kg, 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. Administration of a combination of vitamers and bioactive compounds can be continuous. Administration of a combination of vitamers and bioactive compounds can be intermittent.

For example, a composition, comprising vitamers and bioactive compounds, can be administered at an amount of about: 1 μg/kg to about 200 mg/kg, 10 μg/kg to about 150 mg/kg, 100 μg/kg to about 100 mg/kg, 1 mg/kg to about 50 mg/kg, 1 mg/kg to about 25 mg/kg, 1 mg/kg to about 10 mg/kg, 1 mg/kg to about 5 mg/kg, or 1 mg/kg to about 2 mg/kg, QD. For example, a composition, comprising vitamers and bioactive compounds, can be administered at an amount of about: 1 μg/kg to about 200 mg/kg, 10 μg/kg to about 150 mg/kg, 100 μg/kg to about 100 mg/kg, 1 mg/kg to about 50 mg/kg, 1 mg/kg to about 25 mg/kg, 1 mg/kg to about 10 mg/kg, 1 mg/kg to about 5 mg/kg, or 1 mg/kg to about 2 mg/kg, BIW. For example, a composition, comprising vitamers and bioactive compounds, can be administered at an amount of about: 1 μg/kg to about 200 mg/kg, 10 μg/kg to about 150 mg/kg, 100 μg/kg to about 100 mg/kg, 1 mg/kg to about 50 mg/kg, 1 mg/kg to about 25 mg/kg, 1 mg/kg to about 10 mg/kg, 1 mg/kg to about 5 mg/kg, or 1 mg/kg to about 2 mg/kg, TIW. For example, a composition, comprising vitamers and bioactive compounds, can be administered at an amount of about: 1 μg/kg to about 200 mg/kg, 10 μg/kg to about 150 mg/kg, 100 μg/kg to about 100 mg/kg, 1 mg/kg to about 50 mg/kg, 1 mg/kg to about 25 mg/kg, 1 mg/kg to about 10 mg/kg, 1 mg/kg to about 5 mg/kg, or 1 mg/kg to about 2 mg/kg, QW. For example, a composition, comprising vitamers and bioactive compounds, can be administered at an amount of about: 1 μg/kg to about 200 mg/kg, 10 μg/kg to about 150 mg/kg, 100 μg/kg to about 100 mg/kg, 1 mg/kg to about 50 mg/kg, 1 mg/kg to about 25 mg/kg, 1 mg/kg to about 10 mg/kg, 1 mg/kg to about 5 mg/kg, or 1 mg/kg to about 2 mg/kg, Q2W. In one example, a combination of vitamers and bioactive compounds can be administered at an amount of about 15 mg/kg to about 75 mg/kg, QD. In another example, a combination of vitamers and bioactive compounds can be administered at an amount of about 20 mg/kg to about 50 mg/kg. In still another example, a combination of vitamers and bioactive compounds can be administered at an amount of about 0.00001 mg/kg, 0.0001 mg/kg, 0.001 mg/kg, 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. Administration of a combination of vitamers and bioactive compounds can be continuous. Administration of a combination of vitamers and bioactive compounds can be intermittent.

As used herein, the term daily is intended to mean that a therapeutic compound of a combination described herein, such as a combination of vitamers and bioactive compounds, is administered once or more than once each day for a period of time. The term continuous is intended to mean that a therapeutic compound of a combination described herein, such as a combination of vitamers and bioactive compounds, is administered daily for an uninterrupted period of at least 10 days to 52 weeks. The term intermittent or intermittently as used herein is intended to mean stopping and starting at either regular or irregular intervals. For example, intermittent administration of a therapeutic compound of a combination described herein, such as a combination of vitamers and bioactive compounds, includes administration for one to six days per week (e.g., 2 to 3 times per week or QD), administration in cycles (e.g., daily administration for two to eight consecutive weeks, then a rest period with no administration at least one day), or, for example, administration on alternate days.

Where the combination described herein includes a bioactive compound, it can be administered according to established regimens such as those provided in a package insert. The bioactive compound can be administered in an amount described herein and can be administered QW, once every 2 weeks (Q2W), once every 3 weeks (Q3W), or once every 4 weeks (Q4W). In one embodiment, the bioactive compound is administered Q2W or Q4W. In another embodiment, the bioactive compound is administered Q2W. In yet another embodiment, the bioactive compound is administered Q3W. In still another embodiment, the bioactive compound is administered BIW for at least 3 weeks. In still another embodiment, the bioactive compound is administered Q4W.

The combination of vitamers and bioactive compounds can be administered as an intravenous infusion over about 10, 20, 30, 40, 50, or 60 or more minutes. the combination of vitamers and bioactive compounds can be administered as an intravenous infusion over about 60 minutes once every 1, 2, 3, 4, 5 or more weeks. The combination of vitamers and bioactive compounds can be administered as an intravenous infusion over about 60 minutes once every two weeks. The combination of vitamers and bioactive compounds can be administered as an intravenous infusion over about 60 minutes once every three weeks. The combination of vitamers and bioactive compounds can be administered as an intravenous infusion over about 60 minutes once every four weeks. The combination of vitamers and bioactive compounds can be administered as an intravenous infusion according to a package insert. Administration of the combination of vitamers and bioactive compounds can be continuous. Administration of the combination of vitamers and bioactive compounds can be intermittent.

The combinations described herein can be administered in a regimen. For example, the regimen can be structured to provide therapeutically effective amounts of a combination of vitamers and bioactive compounds, over a predetermined period of time (e.g., an administration time). The regimen can be structured to limit or prevent side-effects or undesired complications of each of the components of the combination described herein. The regimen can be structured in a manner that results in increased effect for both therapies of the combination (e.g., synergy). Regimens useful for treating the indications provided herein can include any number of days of administration which can be repeated as necessary. Administration periods can be broken by a rest period that includes no administration of at least one therapy. For example, a regimen can include administration periods that include 2, 3, 5, 7, 10, 15, 21, 28, or more days. These periods can be repeated. For example, a regimen can include a set number of days as previously described where the regimen is repeated 1, 2, 3, 4, 5, 6, 7, 8, 9, 10, 11, 12, 13, or more times.

Regimens can include a rest period of at least 1, 2, 3, 5, 7, 10, or more days, where at least one therapy is no longer administered to a patient. The rest period can be determined by, for example, monitoring the reaction of the patient to the drug or by measuring the efficacy of the treatment. A rest period can be applicable to a single therapy, such that only one therapy of a combination described herein is discontinued in the rest period but the other therapy(ies) are still administered. Rest periods can be applied to all of the therapies administered to the subject such that the subject receives no therapy for a set period of time during the rest period.

Regimens described herein for treatments or therapies using the combinations described herein can be continued until disease progression or unacceptable toxicity.

Regimens for administration of combinations described herein include, for example administration of vitamers, plus a bioactive compound. For example, a combination of vitamers can be administered QD for about 21 days and a bioactive compound described herein can be administered Q2W or Q4W). For example, a combination of vitamers can be administered BIW or TIW and a bioactive compound described herein can be administered Q2W. In another exemplary regimen, a combination of vitamers can be administered BIW or TIW and a bioactive compound can be administered BIW for 2 or 3 weeks. In still another exemplary regimen, a combination of vitamers can be administered BIW or TIW and a bioactive compound can be administered Q4W. In still another exemplary regimen, a combination of vitamers can be administered BIW and a bioactive compound described herein can be administered Q2W, Q3W, or Q4W. In certain embodiments, such regimens include administration of a bioactive compound administered Q2W, Q3W, or Q4W. In yet another exemplary regimen, a combination of vitamers can be administered TIW and a bioactive compound described herein can be administered Q2W, Q3W, or Q4W. In certain embodiments, such regimens include administration of a bioactive compound administered Q2W, Q3W, or Q4W. In certain embodiments, such regimens include administration of a combination of vitamers administered QD. In certain embodiments, such regimens include administration of a combination of vitamers administered QD for at least 21 days. In yet another exemplary regimen, a combination of vitamers can be administered QD or QW and a bioactive compound is administered Q2W, Q3W, or Q4W.

The regimen can be a regimen for administration of a bioactive compound with a combination of vitamers as described herein. In one exemplary regimen including a bioactive compound, a combination of vitamers can be administered BIW or TIW and a bioactive compound is administered in accordance with the prescribing information provided in, for example, a package insert. In another exemplary regimen, a bioactive compound is administered at an amount of about 1 mg/kg to about 20 mg/kg on day 1 of the regimen, and Q2W thereafter until disease progression or unacceptable toxicity and a combination of vitamers is administered BIW or TIW over the same period of time. In another exemplary regimen, a bioactive compound is administered at an amount of about 1 mg/kg to about 20 mg/kg on day 1 of a regimen, and Q3W thereafter until disease progression or unacceptable toxicity and a combination of vitamers is administered BIW or TIW over the same period of time. A bioactive compound can be administered Q4W with a combination of vitamers, where the combination of vitamers is administered, for example, BIW or TIW during the course of such a regimen. A bioactive compound can be administered Q2W with a combination of vitamers, where the combination of vitamers is administered, for example, BIW or TIW during the course of such a regimen. In still another exemplary regimen, a bioactive compound can be administered Q2W or Q4W with a combination of vitamers, where the combination of vitamers is administered, for example, QD or QW during the course of such a regimen. Such regimens can be repeated as described above (e.g., 1, 2, 3, 4, 5, 6, 7, 8, 9, 10, 11, 12, or more times).

In another exemplary regimen including a bioactive compound, a combination of vitamers can be administered QD and a bioactive compound is administered in accordance with the prescribing information provided in, for example, a package insert. In another exemplary regimen, a bioactive compound is administered at an amount of about 1 mg/kg to about 20 mg/kg on day 1 of the regimen, and Q2W thereafter until disease progression or unacceptable toxicity and a combination of vitamers is administered QD over the same period of time. In another exemplary regimen, a bioactive compound is administered at an amount of about 1 mg/kg to about 20 mg/kg on day 1 of a regimen, and Q3W thereafter until disease progression or unacceptable toxicity and a combination of vitamers is administered QD over the same period of time. A bioactive compound can be administered Q4W with a combination of vitamers, where the combination of vitamers is administered QD during the course of such a regimen. A bioactive compound can be administered Q2W with a combination of vitamers, where the combination of vitamers is administered QD during the course of such a regimen. Such regimens can be repeated as described above (e.g., 1, 2, 3, 4, 5, 6, 7, 8, 9, 10, 11, 12, or more times).

It should also be appreciated that the combinations described herein for treating the indications provided herein can be co-administered with other active agents other than those present in the combinations described herein (e.g., anti-cancer agents). Regimens for administration of a combination described herein, including the exemplary regimens set forth above, can be modified as necessary to include administration of such active agents. Administration of such active agents, e.g., anti-cancer agents, can be performed QD, QW, QM, BID, BIW, TIW, Q2W, Q3W, or Q4W, or in accordance with prescribing information for such anti-cancer agents as set forth, for example, in a package insert.

It is understood that modifications which do not substantially affect the activity of the various embodiments of this invention are also included within the definition of the invention provided herein.

### Formulations

The three vitamer ingredients described herein can be the basis for an OTC (over-the- counter) multivitamer supplement or a prescribed version. A separate label of the same ingredients can be labeled for prescription pre-natal use. The preferred dosage ranges for three bioactive vitamers in claimed compositions are as follows: L-Methylfolate from 500 μg to 1500 μg, Adenosylcobalamin from 1000 μg to 2500 μg, Methylcobalamin from 1000 μg to 2500 μg. In certain embodiments, dosage of L-Methylfolate in claimed compositions can be 300 μg, 500 μg, 750 μg, 900 μg, 1000 μg, 1200 μg, 1500 μg, 2000 μg, 3000 μg or higher. In certain embodiments, dosage of Adenosylcobalamin in claimed compositions can be 500 μg, 750 μg, 1000 μg, 1200 μg, 1500 μg, 2000 μg, 2500 μg, 3000 μg, 4000 μg, 5000 μg or higher. In certain embodiments, dosage of Methylcobalamin in claimed compositions can be 500 μg, 750 μg, 1000 μg, 1200 μg, 1500 μg, 2000 μg, 2500 μg, 3000 μg, 4000 μg, 5000 μg or higher.

The formulation of these three vitamers can be added to a bioactive compound (medication) to complete an OTC form or a prescribed form. In some embodiments, the three bioactive vitamers taken once a day is an OTC medication. In some embodiments, the same tablet taken twice a day requires a prescription, depending on a dosage.

In certain embodiments, the therapeutically effective amount of an Inhibitor Antibody is determined as an amount provided in a package insert provided with the Inhibitor Antibody. The term package insert refers to instructions customarily included in commercial packages of medicaments approved by the FDA or a similar regulatory agency of a country other than the USA, which contains information about, for example, the usage, dosage, administration, contraindications, and/or warnings concerning the use of such medicaments.

### EXAMPLES

Aspects of the present teachings may be further understood in light of the following examples, which should not be construed as limiting the scope of the present teachings in any way.

### Example 1—Enhanced Immune Response

This following example describes use of bioactive B12/B9 vitamins as vaccine adjuvants to increase immunocompetence. The body's ability to manifest a competent immune response to vaccines is dependent on the micronutrient status of the individual. The most important micronutrients for the development of a comprehensive antibody response to vaccines are the vitamins B12 and B9. Many individuals have subclinical deficiency of these nutrients due to various factors, such as increased BMI, age, gender, alcohol consumption, MTHFR deficiency, NO2 exposure, diabetes, veganism, and/or prescribed use of various medications. This large population of immune-incompetent individuals is at risk of a sub-therapeutic immune response to all vaccines because of low levels of B12 or B9.

To facilitate the maximum immune response in these individuals, it is proposed that these B-vitamins be directly added to the intramuscular injection vaccine fluid with the specification that two weeks' worth of an oral b.i.d. dosing of B12 and B9 be dispensed for follow each vaccination.

Without being bound to a particular theory, it is understood that seniors suffer from an age-related decrease in the ability to absorb B12 from protein while simply ingesting less. Other populations are also at risk for a reduced immune response to vaccinations, i.e. those that are obese, diabetics taking Metformin (GLUCOPHAGE®), patients taking GERD medications, women taking birth control pills or hormone replacement therapy. The study below shows how low levels of B12 weakens the immune reaction by suppressing the antibody response to vaccinations. In essence, the vaccine is not providing the benefits doctor and patient believe, leaving the bacteria to strike with deadly force. The loss in lives and healthcare funds is enormous and could be prevented with just a few dollars' worth of preventative B12 and folate. See, Fata, FT, et al, “Impaired Antibody Responses to Pneumococcal Polysaccharide in Elderly Patients with Low Serum Vitamin B12 Levels,” Annals of Internal Medicine, Feb. 1, 1996, 124(3):299-304. This study below shows that methylcobalamin improves the antibody response of the immune system (“We conclude that vit. B12 acts as an immunomodulator for cellular immunity.”). In other examples, the studies below shows an improved H1N1 influenza vaccine response in mothers when supplemented with B12 (Tamura J, et al. Immunomodulation by vitamin B12: augmentation of CD8+T lymphocytes and natural killer (NK) cell activity in vitamin B12-deficient patients by methyl-B12 treatment. Clin Exp Immunol. 1999 April; 116(1):28-32; Siddiqua T J, et. al. Vitamin B12 supplementation during pregnancy and postpartum improves B12 status of both mothers and infants but vaccine response in mothers only: a randomized clinical trial in Bangladesh. Eur J Nutr. 2016 February; 55(1):281-93).

### Example 2—Antidepressants

An established method of determining the effect of administered antidepressants can be found under Hre, Z & Rk, S & Saka, Cafer & Te, I. (2011). Analytical methods for determination of selective serotonin reuptake inhibitor antidepressants. Reviews in Analytical Chemistry. 30. 87-122. 10.1515/REVAC.2011.018. One of skill in the art will be able to determine the comparative or synergistic effect of the combinations described herein with the antidepressant administered alone.

L-methylfolate, under the brand name DEPLIN®, is classified as a FDA-approved medicinal food for antidepressant augmentation. L-methylfolate is the only ingredient in the prescription medicinal food DEPLIN®, which has an FDA indication as an antidepressant augmentation agent. Mentioned in the study below is the fact that 56% of depressed people in one study had a folate deficiency which explains why there is such a positive response to adding folate to antidepressants. B12 and folate also work by increasing the natural production of neurotransmitters and increase the thickness of myelin, which covers and protects nerve cell connections. See, Marian W. Roman & Fay H. Bembry (2011) L-Methylfolate (Deplin®): A New Medical Food Therapy as Adjunctive Treatment for Depression, Issues in Mental Health Nursing, 32:2, 142-143.

The following study revealed that patients showed “significant improvements in self-reported depression symptoms and functioning and greater satisfaction with their medication treatment.” Shelton R C, Sloan Manning J, Barrentine L W, Tipa E V. Assessing Effects of 1-Methylfolate in Depression Management: Results of a Real-World Patient Experience Trial. Prim Care Companion CNS Disord. 2013; 15(4):PCC.13m01520. doi:10.4088/PCC.13m01520.

The most common antidepressants useful in this invention include, but are not limited to, fluoxetine (PROZAC®) (SARAFEM®), citalopram (CELEXA®), sertraline (ZOLOFT®), paroxetine (PAXIL®), escitalopram (LEXAPRO®), Bupropion (WELLBUTRIN®) (ZYBAN®), venlafaxine (EFFEXOR XR®), duloxetine (CYMBALTA®), and fluvoxamine (LUVOX®).

Classes of antidepressants useful in the invention include Selective serotonin reuptake inhibitors (SSRIs), Serotonin-norepinephrine reuptake inhibitors (SNRIs), Tricyclic antidepressants (TCAs), Monoamine oxidase inhibitors (MAOIs), and Atypical agents.

### Example 3—Antipsychotics

An established method of determining the effect of administered antipsychotics can be found under the protocol titled “Clinical Antipsychotic Trials of Intervention Effectiveness (CATIE)” found at the US National Institutes of Health web site at https://www.nimh.nih.gov/funding/clinical-research/practical/catie/index.shtml (last accessed Apr. 30, 2021). One of skill in the art will be able to determine the comparative or synergistic effect of the combinations described herein with the antipsychotic administered alone.

Folate and B12 improve response to antipsychotics by reducing the waste byproduct homocysteine which worsens symptoms and optimizing nerve cell function. Like all the other adjunct examples, the presence of optimal B vitamers reduces risk of illness and a deficiency increases the risk. In essence, B12/F deficiency worsens mental health and neurologic problems in five ways:
Myelin production decreases, and the nerve cells communicate inefficiently.
Homocysteine increases, increasing inflammation in the brain.
MAO released from the immature platelets of deficiency shortens the life of neurotransmitters so messages between brain cells are slowed.
DNA-methylation is reduced so there is a reduction in the coordinated expression of the proteins, leading to poor functioning and aging of the brain cells.
Neurotransmitter production decreases because the precursor SAMe can't be made.
   See, Joshua L. Roffman et al. Randomized Multicenter Investigation of Folate Plus Vitamin B12 Supplementation in Schizophrenia. JAMA Psychiatry, 2013 (“. . . folate plus vitamin B12 supplementation can improve negative symptoms opens a new potential avenue for treatment of schizophrenia.”); Muntjewerff J W, et.al Homocysteine, methylenetetrahydrofolate reductase and risk of schizophrenia: a meta-analysis. Mol Psychiatry. 2006 February; 11(2):143-9 (“The elevated risk of schizophrenia associated with the homozygous genotype of the MTHFR 677C>T polymorphism provides support for causality between a disturbed homocysteine metabolism and risk of schizophrenia.”).

The following study indicates that when there is a decrease in serum folate, there is an increase in the risk of psychosis (“decreased plasma folate may act as a risk factor for schizophrenia.”) Saedisomeolia A, Djalali M, Moghadam A M, Ramezankhani O, Najmi L. Folate and vitamin B12 status in schizophrenic patients. J Res Med Sci. 2011; 16 Suppl 1(Supp11): 5437-5441.

The following case study paper specifically addresses B12 deficiency in psychosis (“mood disorders with psychotic features especially with accompanying extrapyramidal symptoms lacking a clear etiology may be rare manifestation of vitamin B12 and/or folate deficiency.”). Tufan A E, Bilici R, Usta G, Erdo\check{g}an A. Mood disorder with mixed, psychotic features due to vitamin b12 deficiency in an adolescent: case report. Child Adolesc Psychiatry Ment Health. 2012; 6(1):25. Published 2012 Jun. 22.

The most common antipsychotics useful in the present invention include, but are not limited to, Haloperidol, loxapine, thioridazine, molindone, thiothixene, fluphenazine. mesoridazine, trifluoperazine, perphenazine, chlorpromazine, aripiprazole, clozapine, ziprasidone, risperidone, quetiapine, and olanzapine.

### Example 4—Diabetes Therapies

Diabetic therapies reduce blood sugar down toward the normal range. The one most often prescribed, Metformin (GLUCOPHAGE®), has an excellent safety profile for treating type 2 diabetes, but it is associated with risks. It has the negative side effect of reducing the ability to utilize vitamin B12. This vitamin deficiency contributes to the development of a wide variety of medical illnesses besides directly causing anemia and nerve damage (neuropathy).

It is important to note that when considering adding vitamers to diabetes medication is that one formulation (METANX®) already has FDA approval for treating diabetic neuropathy. However, the combinations of the present invention are novel and surprisingly result in the enhanced effectiveness of Metformin administration. To restate, the treatment that reduces the side-effects of the medication also treats one of the most severe co-morbid conditions associated with diabetes—nerve damage leading to amputation.

When diabetic therapies cause vitamin a B12 deficiency an increase in serum homocysteine occurs. This cell waste product causes endothelial inflammation and increased blood viscosity leading to heart attacks, strokes and pulmonary emboli. See, Shargorodsky, M., & Omelchenko, E. (2017). Do adding B vitamins and folate to Metformin has beneficial effect on renal function and lipids in type 2 diabetic patients. Endocrine Abstracts (“Adding B vitamins and folate supplementations to metformin was associated with beneficial effects on HDL cholesterol and renal function in diabetic patients.”); M G Wulffelé, et. al Effects of short-term treatment with metformin on serum concentrations of homocysteine, folate and vitamin B12 in type 2 diabetes mellitus: a randomized, placebo-controlled trial. J Intern Med 2003 November; 254(5):455-63 (“In patients with type 2 diabetes, 16 weeks of treatment with metformin reduces levels of folate and vitamin B12, which results in a modest increase in homocysteine.”); Pawlak R (2017) Vitamin B12 for Diabetes Patients Treated with Metformin. J Fam Med Dis Prey 3:057 (“Metformin treatment, both duration and dose, is associated with increased risk of vitamin B12 (B12) deficiency. B12 deficiency causes Hyperhomocysteinemia (HHcy), which is associated with an increased risk of a variety of diabetic co-morbidities.”); and Xu L, Huang Z, He X, Wan X, Fang D, Li Y. Adverse effect of metformin therapy on serum vitamin B12 and folate: short-term treatment causes disadvantages? Med Hypotheses. 2013 August; 81(2):149-51 (“Recent observational studies, however, have demonstrated that long-term metformin therapy increases the probability of vitamin B12 and folate deficiency, and might contribute to the progression of diabetic peripheral neuropathy.”).

### Example 5—Cancer Therapies

Those of skill in the art will recognize that anti-cancer medications, such as methotrexate (which is an anti-folate agent) are not suitable to use in the present invention. However, it is clear that vitamers can prevent cancer in several other ways (see the references below). For example, Susanna C., et al, “Folate Intake, MTHFR Polymorphisms, and Risk of Esophageal, Gastric, and Pancreatic Cancer: A Meta-analysis,” Gastroenterology, Volume 131, Issue 4, October 2006, 1271-1283 and Kono S, Chen K. Genetic polymorphisms of methylenetetrahydrofolate reductase and colorectal cancer and adenoma. Cancer Sci. 2005 September; 96(9):535-42 provide that “epidemiological findings regarding MTHFR C677T polymorphism provide strong evidence that adequate folate status confers protection from colorectal cancer.” L L Wu and J T Wu , “Hyperhomocysteinemia Is a Risk Factor for Cancer and a New Potential Tumor Marker,” Clinica Chimica Acta., 2002 August; 322(1-2):21-8 provide that “they [homocysteine] may be used as a more accurate tumor marker for monitoring cancer patients during treatment, and hyperhomocysteinemia as a risk factor for carcinogenesis.”

### A. Response Criteria

RECIST is a set of established criteria or standards, internationally recognized for evaluating patient response, stability and progression in clinical trials and in the clinical practice. Originally published in 2000, and revised in 2009 (Eisenhauer E A, et al.; New response criteria in solid tumors: revised RECIST guideline (version 1.1); Eur J Cancer 2009; 45:228-47), as a joint effort of the European Organization for Research and Treatment of Cancer, the National Cancer Institute of the United States and the National Cancer Institute of Canada Clinical Trials Group, RECIST has traditionally been utilized in the evaluation of response to chemotherapy.

### Evaluation of Target Lesions

Complete Response (CR): Disappearance of all target lesions; Partial Response (PR): At least a 30% decrease in the sum of the LD (longest diameter) of target lesions, taking as reference the baseline sum LD; Stable Disease (SD): Neither sufficient shrinkage to qualify for PR nor sufficient increase to qualify for PD, taking as reference the smallest sum LD since the treatment started; Progressive Disease (PD): At least a 20% increase in the sum of the LD of target lesions, taking as reference the smallest sum LD recorded since the treatment started or the appearance of one or more new lesions.

### Evaluation of Non-Target Lesions

Complete Response (CR): Disappearance of all non-target lesions and normalization of tumor marker level; Incomplete Response/Stable Disease (SD): Persistence of one or more non-target lesion(s) or/and maintenance of tumor marker level above the normal limits; Progressive Disease (PD): Appearance of one or more new lesions and/or unequivocal progression of existing non-target lesions.

### Other Response Criteria

Other response criteria include the Immune-Related Response Criteria or iRECIST, as defined by Wolchok et al., in 2009 (Wolchok J D, et al.; Guidelines for the Evaluation of Immune Therapy Activity in Solid Tumors: Immune-Related Response Criteria. Clin Cancer Res 2009; 15(23):7412-20) and the revised International Working Group Response Criteria (Cheson B D et al. Revised response criteria for malignant lymphoma. J. Clin. Oncol. 2007; 25:579-586).

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### References Cited

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A composition comprising a therapeutically effective amount of: 1) L-Methylfolate, 2) Adenosylcobalamin, 3) Methylcobalamin, and 4) at least one bioactive compound or a pharmaceutically acceptable salt of the at least one bioactive compound, wherein the bioactive compound does not comprise a vitamin or an elemental metal.

2. The composition of claim 1, wherein the bioactive compound comprises an active ingredient of a drug or an active component of a vaccine.

3. The composition of claim 2, wherein the drug is selected from the group consisting of a diabetes therapeutic, gastric acidity therapeutic, hormone replacement therapeutic, oral contraceptive, corticosteroid, antibiotic, antiseizure therapeutic, tumor vaccines, HIV therapeutic, antidepressant, cholesterol therapeutic, gout therapeutic, blood pressure therapeutic, osteoporosis therapeutic, antipsychotic therapeutic, constipation therapeutic, anti-Parkinson's therapeutic, pain therapeutic, oral therapeutic, topical therapeutic, wound therapeutic, nutritional aid, cancer therapeutic, infertility therapeutic, dementia therapeutic, macular degeneration therapeutic, fibromyalgia therapeutic, and any combination thereof.

4. The composition of claim 2, wherein the composition comprises L-Methylfolate from 500 μg to 1500 μg, Adenosylcobalamin from 1000 μg to 2500 μg and Methylcobalamin from 1000 μg to 2500 μg.

5. The composition of claim 3, wherein the diabetes therapeutic is selected from the group consisting of insulin, oral antihyperglycemic drugs, sulfonylureas, short-acting insulin secretagogues, biguanides, thiazolidinediones, alpha-glucosidase inhibitors, dipeptidyl peptidase-4 inhibitors, sodium-glucose co-transporter 2 inhibitors, dopamine agonist, injectable antihyperglycemic drugs, glucagon-like peptide-1 (GLP-1) receptor agonists, amylin analog, adjunctive drug therapy, Chlorpropamide, Dapagliflozin, Canagliflozin, Ertugliflozin, Bromocriptin, Empagliflozin, Rosiglitazone, Lixisenatide, Pioglitazone, Repaglinide, liraglutide, Glimepiride, Linagliptin, albiglutide, Tolbutamide, pramlintide, epinephrine, Saxagliptin, Semaglutide, Sitagliptin, dulaglutide, Nateglinide, Alogliptin, Metformin, Glipizide, exenatide, Glyburide, Acarbose, Miglitol, and any combination thereof.

6. The composition of claim 3, wherein the gastric acidity therapeutic is selected from the group consisting of proton pump inhibitors, H2 blockers, histamine H2 antagonists, antacids, prostaglandins, sucralfate, sodium bicarbonate, esomeprazole, pantoprazole, lansoprazole, theophylline, tetracycline, rabeprazole, misoprostol, omeprazole, nizatidine, Ranitidine, famotidine, cimetidine, Sucralfate, lidocaine, phenytoin, warfarin, diazepam, digoxin, and any combination thereof.

7. The composition of claim 3, wherein the hormone replacement therapeutic is selected from the group consisting of conjugated equine or synthetic estrogens, estradiol, medroxyprogesterone acetate, progesterone, natural progesterone, synthetic progesterone, norethindrone, drospirenone, levonorgestrel, megastrol, selective estrogen receptor modulators, tamoxifen, raloxifene, bazedoxifene, and any combination thereof.

8. The composition of claim 3, wherein the oral contraceptive therapeutics is selected from the group consisting of gonadotropin-releasing hormone inhibitors, pituitary hormone inhibitors, estradiol, ethinyl estradiol, estrogen with progestin, estrogen without progestin, progestins, Norgestimate, etonogestrel, desogestrel, levonorgestrel, norethindrone, norethindrone acetate, ethynodiol diacetate, dienogest, drospirenone, progesterone, and any combination thereof.

9. The composition of claim 3, wherein the corticosteroid therapeutic is selected from the group consisting of Class I corticosteroids, Class II corticosteroids, Class III corticosteroids, Class IV corticosteroids, Class V corticosteroids, Class VI corticosteroids, Class VII corticosteroids, Betamethasone dipropionate, Betamethasone valerate, Clobetasol propionate, Diflorasone diacetate, Halobetasol propionate, Amcinonide, Desoximetasone, Fluocinonide, Fluocinolone acetonide, Halcinonide, Mometasone furoate, Fluticasone propionate, Triamcinolone acetonide, Flurandrenolide, Desonide, Hydrocortisone, Hydrocortisone acetate, Hydrocortison acetate and pramoxine hydrochloride, Hydrocortisone butyrate, Hydrocortisone valerate, Alclometasone dipropionate, Flumethasone pivalate, hydrocortisone types, acetonides and related substances, betamethasone types, esters, halogenated esters, labile prodrug esters, and any combination thereof.

10. The composition claim 3, wherein the antibiotics is selected from the group consisting of sulfa drugs, cephalosporins, macrolides, penicillin derivatives, quinolones, tetracycline derivatives, aminoglycosides, trimethoprim, quinupristin/dalfopristin, piperacillin/tazobactam, Penicillin G procaine, clarithromycin, nitrofurantoin, ciprofloxacin, telithromycin, Metronidazole, levofloxacin, erythromycin, Theophylline, Gemifloxacin, Tetracycline, azithromycin, Delafloxacin, Eravacycline, moxifloxacin, Dalbavancin, Amoxicillin, Fidaxomicin, Tigecycline, ceftriaxone, Minocycline, Rifapentine, Clindamycin, Ceftazidime, Oritavancin, Norfloxacin, Doxycycline, Cefuroxime, Tobramycin, Ceftibuten, Gentamicin, Cefotaxime, Vancomycin, Telavancin, Daptomycin, Cephalexin, Fosfomycin, tedizolid, Aztreonam, Nafcillin, Phenytoin, Ertapenem, Cefazolin, Isoniazid, Doripenem, rifabutin, Meropenem, Linezolid, ofloxacin, Cefoxitin, Oxacillin, Warfarin, Neomycin, Rifampin, Cefepime, Digoxin, and any combination thereof.

11. The composition of claim 3, wherein the antiseizure therapeutic is selected from the group consisting of broad-spectrum antiseizure drugs, focal-onset seizure drugs, generalized-onset tonic-clonic seizure drugs, epileptic spasms drugs, atonic seizures drugs, myoclonic seizures drugs, juvenile myoclonic epilepsy drugs, febrile seizure drugs, drugs for seizures due to alcohol withdrawal, anticonvulsants, Eslicarbazepine, Levetiracetam, oxcarbazepine, Acetazolamide, Carbamazepine, phenobarbital, Fosphenytoin, Ethosuximide, lamotrigine, Zonisamide, lacosamide, topiramate, vigabatrin, clonazepam, pregabalin, gabapentin, felbamate, phenytoin, Ezogabine, tiagabine, clobazam, mephobarbital, primidone, valproic acid, and any combination thereof.

12. The composition of claim 2, wherein the vaccine is selected from the group consisting of Adenovirus; Anthrax, including but not limited to AVA; Cholera, including but not limited to Vaxchora; Diphtheria, including but not limited to DTaP, Td, DT, Tdap, DTaP-IPV, DTaP-HepB-IPV, DTaP-IPV/Hib; Hepatitis A, including but not limited to HepA, HepA-HepB; Hepatitis B including but not limited to HepB, DTaP-HepB-IPV, HepA-HepB; Haemophilus influenzae type b (Hib) including but not limited to Hib, DTaP-IPV/Hib;
Human Papillomavirus (HPV) including but not limited to HPV9/9vHPV; Seasonal Influenza (Flu) including but not limited to IIV, LAIV; Japanese Encephalitis including but not limited to JE; Measles including but not limited to MMR, MMRV; Meningococcal including but not limited to MenACWY, MenB; Mumps including but not limited to MMR, MMRV; Pertussis including but not limited to DTaP, Tdap, DTaP-IPV, DTaP-HepB-IPV, DTaP-IPV/Hib; Pneumococcal including but not limited to PCV13, PPSV23; Polio including but not limited to Polio, DTaP-IPV, DTaP-HepB-IPV, DTaP-IPV/Hib; Rabies including but not limited to Rabies; Rotavirus including but not limited to RV1, RV5; Rubella including but not limited to MMR, MMRV; Shingles including but not limited to ZVL, RZV; Smallpox including but not limited to Vaccinia; Tetanus including but not limited to DTaP, Td, DT, Tdap, DTaP-IPV, DTaP-HepB-IPV, DTaP-IPV/Hib; Tuberculosis; Typhoid Fever including but not limited to Typhoid Oral, Typhoid Polysaccharide; Varicella including but not limited to VAR, MMRV; Yellow Fever including but not limited to YF; SARS-CoV-2, and any combination thereof.

13. The composition of claim 3, wherein the HIV therapeutic is selected from the group consisting of antiretroviral therapy (ART), antiretrovirals, nucleoside reverse transcriptase inhibitors (NRTIs), nucleotide reverse transcriptase inhibitors (nRTIs), non-nucleoside reverse transcriptase inhibitors (NNRTIs), protease inhibitors (PIs), entry inhibitors (EIs), post-attachment inhibitors, integrase inhibitors, cyclophosphamide, clarithromycin, Fosamprenavir, Emtricitabine, ketoconazole, itraconazole, Elvitegravir, Dolutegravir, Rilpivirine, Enfuvirtide, Bictegravir, Raltegravir, didanosine, Saquinavir, Atazanavir, Lamivudine, Nelfinavir, Doravirine, Zidovudine, Etravirine, Tipranavir, Nevirapine, Ibalizumab, Stavudine, Maraviroc, estradiol, ritonavir, Darunavir, Tenofovir, Efavirenz, methadone, cisapride, warfarin, fentanyl, Abacavir, and any combination thereof.

14. The composition claim 3, wherein the antidepressant therapeutic is selected from the group consisting of selective serotonin reuptake inhibitors (SSRIs), serotonin modulators (5-HT₂ blockers), serotonin-norepinephrine reuptake inhibitors, norepinephrine-dopamine reuptake inhibitors, heterocyclic antidepressants, monoamine oxidase inhibitors (MAOIs), melatonergic antidepressant, ketamine-like drugs, dextromethorphan, tranylcypromine, pseudoephedrine, levomilnacipran, chlorpromazine, desvenlafaxine, Protriptyline, isocarboxazid, carbamazepine, nortriptyline, clomipramine, vortioxetine, theophylline, escitalopram, Trimipramine, desipramine, propranolol, fluvoxamine, venlafaxine, Mirtazapine, amphetamine, paroxetine, sertraline, selegiline, vilazodone, phenelzine, citalopram, fluoxetine, imipramine, meperidine, metoprolol, duloxetine, esketamine, Trazodone, Bupropion, clozapine, ketamine, warfarin, Doxepin, and any combination thereof.

15. The composition of claim 3, wherein the cholesterol therapeutic is selected from the group consisting of statins, bile acid sequestrants, cholestyramine, cholesterol absorption inhibitors, PCSK9 monoclonal antibodies, dietary supplements, drugs for homozygous familial hypercholesterolemia, fibrates, omega-3 fatty acids, Apo CIII inhibitor, nicotinic acid (niacin), cholesterol ester transport protein (CETP) inhibitors, Pitavastatin, Rosuvastatin, Atorvastatin, cyclosporine, Pravastatin, gemfibrozil, Bezafibrate, Fluvastatin, Colesevelam, Simvastatin, Lomitapide, tacrolimus, Mipomersen, alirocumab, evolocumab, Lovastatin, Colestipol, Metformin, Ezetimibe, warfarin, digoxin, niacin, and any combination thereof.

16. The composition of claim 3, wherein the gout therapeutic is selected from the group consisting of nonsteroidal anti-inflammatory drugs (NSAIDs), corticosteroids, uriosuric therapy, uriosuric agents, clarithromycin, mercaptopurine, nitroglycerin, acetazolamide, azathioprine, prednisolone, cyclosporine, allopurinol, pegloticase, prednisone, colchicine, probenecid, febuxostat, losartan, anakinra, and any combination thereof.

17. The composition of claim 3, wherein the blood pressure therapeutic is selected from the group consisting of adrenergic modifiers, central alpha-2-agonists, postsynaptic alpha-1-blockers, peripheral-acting adrenergic blockers, angiotensin-converting enzyme (ACE) inhibitors, angiotensin II receptor blockers (ARBs), beta-blockers, calcium channel blockers, dihydropyridines, nondihydropyridines, direct renin inhibitors, direct vasoldilators, diuretics, loop diuretics, potassium-sparing diuretics, thiazide-type diuretics, Perindopril erbumine, perindopril, Hydrochlorothiazide, Methyclothiazide, Ethacrynic acid, Spironolactone, chlorthalidone, Chlorothiazide, Trandolapril, triamterene, Candesartan, Telmisartan, Propranolol, Nicardipine, Nisoldipine, Lisinopril, Amlodipine, carvedilol, Furosemide, nifedipine, Benazepril, Irbesartan, Eprosartan, Guanfacine, Felodipine, Olmesartan, acebutolol, metoprolol, Azilsartan, methyldopa, bisoprolol, eplerenone, verapamil, Captopril, amiloride, Quinapril, diltiazem, betaxolol, Enalapril, torsemide, Valsartan, Terazosin, Aliskiren, carteolol, Nebivolol, Doxazosin, minoxidil, Clonidine, Ramipril, Losartan, Prazosin, atenolol, lithium, Nadolol, Timolol, bumetanide, indapamide, hydralazine, fosinopril, moexipril, and any combination thereof.

18. The composition of claim 3, wherein the osteoporosis therapeutic is selected from the group consisting of calcium and vitamin D supplements, antiresorptive drugs, bisphosphonates, hormone replacement therapy, selective estrogen receptor modulators, activator of nuclear facor kappa-B ligand (RANKL) inhibitor, anabolic agents, parathyroid hormone (PTH) analogues, romosozumab, medroxyprogesterone, cholecalciferol, Zoledronic acid, ergocalciferol, abaloparatide, rosiglitazone, teriparatide, pioglitazone, tetracycline, Ibandronate, Alendronate, Risedronate, Raloxifene, calcitriol, calcitonin, denosumab, heparin, and any combination thereof.

19. The composition of claim 3, wherein the antipsychotic therapeutic is selected from the group consisting of dopamine-2 blockers, second-generation psychotics (SGAs), Diphenhydramine, Trihexyphenidyl, norepinephrine, Brexpiprazole, Paliperidone, Aripiprazole, Fluphenazine, Thiothixene, ziprasidone, Iloperidone, Benztropine, risperidone, Cariprazine, haloperidol, Lurasidone, olanzapine, Clonazepam, quetiapine, Lorazepam, clozapine, Loxapine, Pimozide, Lithium, and any combination thereof.

20. The composition of claim 3, wherein the constipation therapeutic is selected from the group consisting of osmotic laxatives, bulking agents, osmotic agents, secretory or stimulant cathartics, enemas, emollient agents, peripherally acting mu-opioid receptor antagonists (PAMORAs), magnesium sulfate, Lubiprostone, Linaclotide, lidocaine, Lactulose, Bisacodyl, senna, miralax, psyllium, calcium polycarbophil, methylcellulose, phenolphthalein, anthraquinones, castor oil, ducosate, mineral oil, lactulose, and any combination thereof.

21. The composition of claim 3, wherein the anti-Parkinson's therapeutic is selected from the group consisting of Carbidopa/levodopa, Amantadine, MAO type B (MOA-B) inhibitors, anticholinergic drugs, dopamine agonists, catechol O-methyltransferase (COMT) inhibitors, oral dopamine agonists, trimethobenzamide, diphenhydramine, Trihexyphenidyl Bromocriptine, Benztropine, risperidone, pramipexole, apomorphine, amphetamine, rasagiline, entacapone, selegiline, olanzapine, rotigotine, quetiapine, ropinirole, tolcapone, clozapine, carbidopa, bisacodyl, levodopa, and any combination thereof.

22. The composition of claim 3, wherein the pain therapeutic is selected from the group consisting of nonopioid analgesics, opioid analgesics, antidepressants, antiseizure drugs, central nervous system (CNS)-active drugs, adjuvant analgesic drugs, nonsteroidal anti-inflammatory drugs (NSAIDs), aspirin, codeine, guaifenesin, hydrocodone, Choline magnesium trisalicylate, Dextroamphetamine, Dextromethorphan, prochlorperazine, diphenhydramine, Methylphenidate, norepinephrine, Metoclopramide, Mefenamic acid, Oxcarbazepine, Hydromorphone, Dexamethasone, Acetaminophen, Buprenorphine, Carbamazepine, nortriptyline, Indomethacin, lubiprostone, testosterone, Flurbiprofen, desipramine, misoprostol, venlafaxine, Hydroxyzine, pentazocine, Pamidronate, tamsulosin, Prednisone, Ketoprofen, pregabalin, Tizanidine, Meperidine, gabapentin, ziconotide, Naltrexone, diclofenac, duloxetine, Fenoprofen, Ibuprofen, bupropion, ketorolac, Capsaicin, lidocaine, Celecoxib, Phenytoin, Modafinil, lactulose, Oxycodone, Memantine, Piroxicam, Methadone, Oxaprozin, bisacodyl, clonidine, naloxone, Tramadol, Sulindac, Naproxen, morphine, Fentanyl, Baclofen, and any combination thereof.

23. The composition of claim 3, wherein the oral therapeutic is selected from the group consisting of toothpaste, dental plaque removers, halitosis suppressing treatments, dental disorder treatments, oral disorder treatments, fluoride, gingivitis treatments, baking soda, salt, lidocaine, saline rinses, topical gum infection agents, floss, whitening treatments, hydrogen peroxide, and any combination thereof.

24. The composition of claim 3, wherein the topical therapeutic is selected from the group consisting of shampoos, conditioners, hair lotions, moisturizers, psoriasis treatments, immunosuppressants, emollients, salicylic acid, coal tar, anthralin, corticosteroids, vitamin D3 analogs, calcineurin inhibitors tazarotene, oral retinoids, cyclosporine, immunomodulatory agents, skin supplements and powders, hair supplements and powders, nail supplements and powder, lip balms, insecticides, keratolytics, astringents, antipruritics, sunscreens, and any combination thereof.

25. The composition of claim 3, wherein the wound therapeutic is selected from the group consisting of bandages, wound healings, burn dressings, disinfectants, sterilizers, antiseptic agents, iodine solutions, and any combination thereof.

26. The composition of claim 3, wherein the nutritional use is selected from the group consisting of vegan meat replacement products, vegan fish replacement products, food fortifications, protein powders, protein bars, nutritional supplements, enteral nutrition, medicinal foods, small and large animal medications, small and large animal feeds, veterinary treatments, infant formulas, breastfeeding alternatives, milk powders, concentrated liquids, prediluted liquids, energy drinks, patches, injections, antifungals, and any combination thereof.

27. The composition of claim 3, wherein the cancer therapeutic is selected from the group consisting of antineoplastic therapy, hormonal therapy, immunotherapy, monoclonal antibodies, interferons, biologic response modifiers, tumor vaccines, cell therapies, retinoids, isocitrate dehydrogenase-2 (IDH2) inhibitors, imatinib, and any combination thereof.

28. The composition of claim 3, wherein the infertility therapeutics is selected from the group consisting of sperm disorder treatments, Medroxyprogesterone, Cyclophosphamide, Spironolactone, Nitrofurantoin, Sulfasalazine, bicalutamide, Ketoconazole, Chlorambucil, cyproterone, Colchicine, Cimetidine, Clomiphene, Estrogens, ovulatory dysfunction treatments, exogenous gonadotropins, progesterone, gonadorelin, metformin, letrozole, decreased ovarian reserve (DOR) treatments, donor oocytes, estradiol, tubal dysfunction and pelvic lesions treatments, abnormal cervical mucus treatments, controlled ovarian stimulation (COS) treatments, urofollitropin, synthetic human chorionic gonadotropin (hCG), follicle stimulating hormone (FSH), methylprednisolone, and any combination thereof.

29. The composition of claim 3, wherein the dementia therapeutic is selected from the group consisting of cholinesterase inhibitors, rivastigmine, benztropine, propranolol, galantamine, memantine, donepezil, and any combination thereof.

30. The composition of claim 3, wherein the macular degeneration therapeutic is selected from the group consisting of lutein, zeaxanthin, antivascular endothelial growth factor (anti-VEGF) drugs, corticosteroids, triamcinolone, bevacizumab, aflibercept, ranibizumab, and any combination thereof.

31. The composition of claim 2, wherein the fibromyalgia therapeutic is selected from the group consisting of tricyclic antidepressants, nonopioid analgesics, cyclobenzaprine, amitriptyline, acetaminophen, milnacipran, bupivacaine, Pregabalin, duloxetine, trazodone, lidocaine, doxepin, and any combination thereof.

32. A method of treating a subject in need thereof with the composition of claim 1, comprising administering the composition of claim 1 to the subject.

33. A kit comprising the composition of claim 1 and an instruction of how to use the composition.
